(19) United States
(12) Patent Application Publication
(10) Pub. No.: US 2005/0277804 A1
(43) Pub. Date: Dec. 15, 2005

(54) System including a cannula having reduced flow resistance

(76) Inventor: Robert Pecor, Aliso Viejo, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/865,045

(22) Filed: Jun. 10, 2004

(51) Int. Cl. A61N 1/362

(52) U.S. Cl. 600/18

(57) ABSTRACT

A cannula system includes a multilumen cannula and a dilator. The multilumen cannula includes a first elongate body and a second elonaget body. The first elongate body has a proximal portion, a distal portion, and a first lumen that extends through the first elongate body. The first lumen is configured to reduce resistance to flow therethrough. The second elongate body defines a second lumen. The second elongate body extends along the proximal portion of the first elongate body. The dilator is configured to extend through the first lumen.

### BACKGROUND OF THE INVENTION

1. Field of the Invention

This application relates to percutaneous cannulae for the introduction and withdrawal of blood from the vascular system and, in particular, to cannulae that are configured to reduce resistance to flow therein.

2. Description of the Related Art

Treatment and diagnosis of a variety of health conditions in a patient can involve withdrawing blood from and returning blood to a patient's vascular system, e.g., in treatment of organ failure. In dialysis treatments, which are sometimes applied to patients suffering from kidney failure, blood is withdrawn from the vascular system, filtered, and infused back into the vascular for further circulation. An emerging treatment for congestive heart failure involves coordinated withdrawal of blood from and infusion of blood into the vascular system. Both such treatments sometimes call for the insertion of cannulae into the vasculature of the patient.

It is sometimes beneficial to access the vascular system by way of a single entry point using a multilumen cannula. Multilumen cannulae enable blood to be withdrawn from the vascular system via a first lumen and infused back into the vascular system via a second lumen. By providing vascular access through a single point, multilumen cannulae are less invasive than other options for coordinated aspiration and infusion, such as the insertion of multiple single lumen cannulae through separate entry sites.

Though multilumen cannulae advantageously can limit the number of entry sites, the size of the lumens of such cannulae are limited by the need to fit more than one lumen into the same region of a vessel. Small lumens can suffer from high flow resistance, especially if relatively long. Increased flow resistance of the lumens of multilumen cannulae present many problems for the devices that are coupled with the cannulae to direct blood into or withdraw blood from the vascular system.

### SUMMARY OF THE INVENTION

Therefore, there is a need for cannulae that reduce the resistance to blood flow in relatively long lumens. Also, there is a need for a percutaneous cannula assembly to enable insertion of such a cannula into the vasculature.

In one embodiment, a multilumen cannula is provided for reducing resistance in blood flowing therethrough. The multilumen cannula has a first elongate portion and a second elongate portion. The first elongate portion defines a first lumen that extends between a first proximal end and a first distal end. The inner cross-section of the first lumen is greater proximate the first distal end than proximate the first proximal end. The second elongate portion defines a second lumen that extends between a second proximal end and a second distal end.

In another embodiment, a multilumen cannula is provided where defines a vessel wall engagement portion and a second lumen that extends between a second proximal end and a second distal end. The inner cross-section of the first lumen is greater at the first distal end than at a location corresponding to the vascular wall engagement portion.

In another embodiment, a multilumen cannula is provided that has a first elongate body and a second elongate body. The first elongate body has a proximal portion, a transition portion, and a distal portion. The transition portion has a diameter at a distal end that is greater than a diameter at a proximal end. The first elongate body also has a first lumen that extends therethrough. The first lumen has a first cross-sectional area within the proximal portion and a second cross-sectional area within the distal portion. The second cross-sectional area is greater than the first cross-sectional area. The second elongate body defines a second lumen. The second elongate body extends along the proximal portion of the first elongate body.

The present invention further comprises a method for accessing the vascular system of a patient. The method comprises the steps of: providing a cannula comprising a first elongate portion and a second elongate portion, the first elongate portion defining a transition portion and a first lumen that extends between a first proximal end and a first distal end, the second elongate portion defining a second lumen that extends between a second proximal end and a second distal end, wherein the inner cross-section of the first lumen is greater proximate the first distal end than proximate a vessel wall engagement portion of said second elongate portion; translating a portion of said cannula to a first position wherein said second distal end is located distal to at least a portion of said transition portion of said first elongate portion; inserting said cannula into a blood vessel through a vascular access site such that said vessel wall engagement portion of said second elongate portion is adjacent said vessel wall; and translating a portion of said cannula to a second position wherein the second distal end is proximal to at least a portion of said transition portion.

In another embodiment, a cannula system is provided that includes a multilumen cannula and a dilator. The multilumen cannula includes a first elongate body and a second elongate body. The first elongate body has a proximal portion, a transition portion distal of the proximal portion, a distal portion distal of the transition portion, and a first lumen. The transition portion has a diameter at a distal end that is greater than a diameter at a proximal end. The first lumen extends through the first elongate body and has a first cross-sectional area within the proximal portion and a second cross-sectional area within the distal portion. The second cross-sectional area is greater than the first cross-sectional area. The second elongate defines a second lumen. The second elongate body extends along the proximal portion of the first elongate body. The dilator is configured to extend through the first lumen.

In another embodiment, a cannula system includes a multilumen cannula and a dilator. The multilumen cannula includes a first elongate body and a second elonaget body. The first elongate body has a proximal portion, a distal portion, and a first lumen that extends through the first elongate body. The first lumen is configured to reduce resistance to flow therethrough. The second elongate body defines a second lumen. The second elongate body extends along the proximal portion of the first elongate body. The dilator is configured to extend through the first lumen.

The present invention further comprises a method for inserting a cannula into a blood vessel. The method comprises: providing a multilumen cannula having a first elongate body and a second elongate body, the first elongate body comprising a proximal portion and a distal portion and defining a first lumen extending therethrough, the first lumen having a larger cross-sectional area in the distal portion than in the proximal portion, the second elongate body defining a second lumen extending therethrough; advancing a dilator into the first lumen in the proximal portion of the first elongate body until a distal portion of the dilator extends distal of the distal portion of the first elongate body; actuating the distal portion of the dilator until the cross-sectional area of the distal portion of the dilator is about equal to the cross-sectional area of the first elongate body at a distal end of the distal portion; and advancing the dilator and the cannula through a percutaneous insertion site and through a vascular insertion site into a blood vessel.

The present invention further comprises a method for treating a patient, comprising: providing a cannula system comprising a multilumen cannula having a first elongate body comprising a proximal portion and a distal portion and defining a first lumen extending therethrough, the first lumen being configured to reduce resistance to flow therethrough, and a second elongate body defining a second lumen and a dilator extending through the first lumen; advancing the cannula system into a blood vessel through a vascular insertion site; providing fluid communication between the second lumen and an inflow port of a pump and between the first lumen and an outflow port of the pump; and operating said pump to perfuse blood through the patient's circulatory system.

### BRIEF DESCRIPTION OF THE DRAWINGS

These and other features and advantages of the invention will now be described with reference to the drawings, which are intended to illustrate and not to limit the invention.

FIG. 1 is a schematic view of one embodiment of a heart assist system having multiple conduits for multi-site application, shown applied to a patient's vascular system;

FIG. 2 is a schematic view of another application of the embodiment of FIG. 1;

FIG. 3 is a schematic view of another embodiment of a heart assist system having multiple conduits for multi-site application wherein each of the conduits is applied to more than one vessel, shown applied to a patient's vascular system;

FIG. 4 is a schematic view of another embodiment of a heart assist system having multiple conduits for multi-site application and employing a connector with a T-shaped fitting, shown applied to a patient's vascular system;

FIG. 5 is a schematic view of an L-shaped connector coupled with an inflow conduit, shown inserted within a blood vessel;

FIG. 6 is a schematic view of another embodiment of a heart assist system having multiple conduits for multi-site application, shown applied to a patient's vascular system;

FIG. 7 is a schematic view of another application of the embodiment of FIG. 6, shown applied to a patient's vascular system;

FIG. 8 is a schematic view of another application of the embodiment of FIG. 6, shown applied to a patient's vascular system;

FIG. 9 is a schematic view of another embodiment of a heart assist system having multiple conduits for multi-site application, a reservoir, and a portable housing for carrying a portion of the system directly on the patient;

FIG. 10 is a schematic view of another embodiment of a heart assist system having a multilumen cannula for single-site application, shown applied to a patient's vascular system;

FIG. 11 is a schematic view of a modified embodiment of the heart assist system of FIG. 10, shown applied to a patient's vascular system;

FIG. 12 is a schematic view of another embodiment of a heart assist system having multiple conduits for single-site application, shown applied to a patient's circulatory system;

FIG. 13 is a schematic view of another application of the embodiment of FIG. 12, shown applied to a patient's vascular system;

FIG. 14 is a schematic view of one application of an embodiment of a heart assist system having an intravascular pump enclosed in a protective housing, wherein the intravascular pump is inserted into the patient's vasculature through a non-primary vessel;

FIG. 15 is a schematic view of another embodiment of a heart assist system having an intravascular pump housed within a conduit having an inlet and an outlet, wherein the intravascular pump is inserted into the patient's vasculature through a non-primary vessel;

FIG. 16 is a schematic view of a modified embodiment of the heart assist system of FIG. 15 in which an additional conduit is shown adjacent the conduit housing the pump, and in which the pump comprises a shaft-mounted helical thread;

FIG. 17 is a schematic view of one embodiment of a multilumen cannula having a variable size lumen;

FIG. 18A is a cross-section view of the multilumen cannula of FIG. 17 taken along section plane 18A-18A;

FIG. 18B is a cross-section view of the multilumen cannula of FIG. 17 taken along section plane 18B-18B;

FIG. 18C is a cross-section view of the multilumen cannula of FIG. 17 taken along section plane 18C-18C;

FIG. 19 is a schematic view of another embodiment of a multilumen cannula having a variable size lumen;

FIG. 19A is a schematic view of another embodiment of a multilumen cannula configured to impart a rotational component to the flow of fluid in a lumen;

FIG. 19B is a cross-sectional view of the multilumen cannula of FIG. 19A taken along section plane 19B-19B;

FIG. 20A is a cross-section view of the multilumen cannula of FIG. 19 taken along section plane 20A-20A;

FIG. 20B is a cross-section view of the multilumen cannula of FIG. 19 taken along section plane 20B-20B;

FIG. 20C is a cross-section view of the multilumen cannula of FIG. 19 taken along section plane 20C-20C;

FIG. 21 is a schematic view of a variation of the embodiment of a multilumen cannula of FIG. 20;

FIG. 22A is a cross-section view of the multilumen cannula of FIG. 21 taken along section plane 22A-22A;

FIG. 22B is a cross-section view of the multilumen cannula of FIG. 21 taken along section plane 22B-22B;

FIG. 22C is a cross-section view of the multilumen cannula of FIG. 21 taken along section plane 22C-22C;

FIG. 23A is a schematic view of another embodiment of a multilumen cannula having a configuration for insertion and a configuration for operation, the configuration for insertion shown;

FIG. 23B is a schematic view of the multilumen cannula of FIG. 23A, showing the configuration for operation;

FIG. 24 is a schematic view of one embodiment of a percutaneous cannula assembly having a dilator configured to facilitate insertion of a multilumen cannula;

FIG. 25 is an exploded view of the percutaneous cannula assembly of FIG. 24;

FIG. 26A is a schematic view of the percutaneous cannula assembly of FIG. 26A partially inserted into the vasculature of a patient;

FIG. 26B is a schematic view of the percutaneous cannula assembly of FIG. 26A inserted into the vasculature of a patient, where the cannula is configured for operation; and

FIG. 27 is a schematic view of a heart-assist system making use of the cannula of FIGS. 23A-23B, where the cannula is configured for operation and in fluid connection with a pump.

### DETAILED DESCRIPTION OF THE PREFERRED EMBODIMENT

Turning now to the drawings provided herein, more detailed descriptions of various embodiments of heart assist systems and cannulae for use therewith are provided below.

I. Extracardiac Heart Assist Systems and Methods

A variety of cannulae and cannula assemblies are described herein that can be used in connection with a variety of heart assist systems that supplement perfusion. Such systems preferably are extracardiac in nature. In other words, the systems supplement blood perfusion, without the need to interface directly with the heart and aorta. Thus, the systems can be applied without major invasive surgery. The systems also lessen the hemodynamic burden or workload on the heart by reducing afterload, impedence, and/or left ventricular end diastolic pressure and volume (preload). The systems also advantageously increase peripheral organ perfusion and provide improvement in neurohormonal status. As discussed more fully below, the systems can be applied using one or more cannulae, one or more vascular grafts, and a combination of one or more cannulae and one or more vascular grafts. For systems employing cannula(e), the cannula(e) can be applied through multiple percutaneous insertion sites (sometimes referred to herein as a multi-site application) or through a single percutaneous insertion site (sometimes referred to herein as a single-site application).

A. Heart Assist Systems and Methods Employing Multi-Site Application

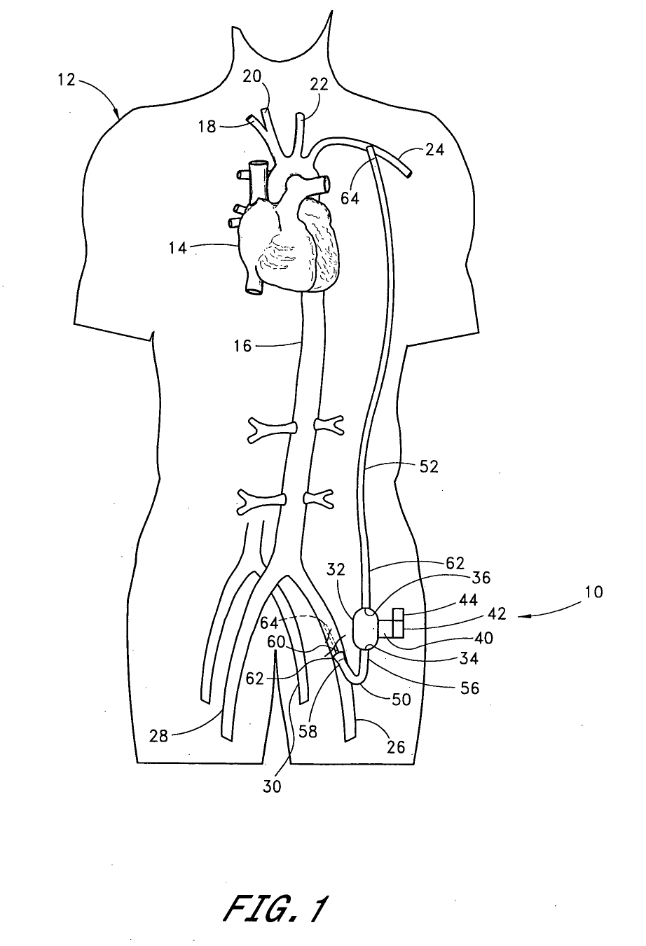

With reference to FIG. 1, a first embodiment of a heart assist system 10 is shown applied to a patient 12 having an ailing heart 14 and an aorta 16, from which peripheral brachiocephalic blood vessels extend, including the right subclavian artery 18, the right carotid artery 20, the left carotid artery 22, and the left subclavian artery 24. Extending from the descending aorta is another set of peripheral blood vessels, the left and right iliac arteries which transition into the left and right femoral arteries 26, 28, respectively. As is known, each of the arteries 16, 18, 20, 22, 24, 26, and 28 generally conveys blood away from the heart. The vasculature includes a venous system that generally conveys blood to the heart. As will be discussed in more detail below, the heart assist systems described herein can also be applied to non-primary veins, including the left femoral vein 30.

The heart assist system 10 comprises a pump 32, having an inlet 34 and an outlet 36 for connection of conduits thereto. The pump 32 preferably is a rotary pump, either an axial type or a centrifugal type, although other types of pumps may be used, whether commercially-available or customized. The pump 32 preferably is sufficiently small to be implanted subcutaneously and preferably extrathoracically, for example in the groin area of the patient 12, without the need for major invasive surgery. Because the heart assist system 10 is an extracardiac system, no valves are necessary. Any inadvertent backflow through the pump 32 and/or through the inflow conduit would not harm the patient 12.

Regardless of the style or nature chosen, the pump 32 is sized to generate blood flow at subcardiac volumetric rates, less than about 50% of the flow rate of an average healthy heart, although flow rates above that may be effective. Thus, the pump 32 is sized and configured to discharge blood at volumetric flow rates anywhere in the range of 0.1 to 3 liters per minute, depending upon the application desired and/or the degree of need for heart assist. For example, for a patient experiencing advanced congestive heart failure, it may be preferable to employ a pump that has an average subcardiac rate of 2.5 to 3 liters per minute. In other patients, particularly those with minimal levels of heart failure, it may be preferable to employ a pump that has an average subcardiac rate of 0.5 liters per minute or less. In yet other patients it may be preferable to employ a pump that is a pressure wave generator that uses pressure to augment the flow of blood generated by the heart.

In one embodiment, the pump 32 is a continuous flow pump which superimposes continuous blood-flow on the pulsatile aortic blood-flow. In another embodiment, the pump 32 has the capability of synchronous actuation; i.e., it may be actuated in a pulsatile mode, either in copulsating or counterpulsating fashion.

For copulsating action, it is contemplated that the pump 32 would be actuated to discharge blood generally during systole, beginning actuation, for example, during isovolumic contraction before the aortic valve opens or as the aortic valve opens. The pump 32 would be static while the aortic valve is closed following systole, ceasing actuation, for example, when the aortic valve closes.

For counterpulsating actuation, it is contemplated that the pump 32 would be actuated generally during diastole, ceasing actuation, for example, before or during isovolumic contraction. Such an application would permit and/or enhance coronary blood perfusion. In this application, it is contemplated that the pump 32 would be static during the balance of systole after the aortic valve is opened, to lessen the burden against which the heart must pump. The aortic valve being open encompasses the periods of opening and closing, wherein blood is flowing therethrough.

It should be recognized that the designations copulsating and counterpulsating are general identifiers and are not limited to specific points in the patient's heart cycle when the pump 32 begins and discontinues actuation. Rather, they are intended to generally refer to pump actuation in which the pump 32 is actuating, at least in part, during systole and diastole, respectively. For example, it is contemplated that the pump 32 might be activated to be out of phase from true copulsating or counterpulsating actuation described herein, and still be synchronous, depending upon the specific needs of the patient or the desired outcome. One might shift actuation of the pump 32 to begin prior to or after isovolumic contraction or to begin before or after isovolumic relaxation.

Furthermore, the pulsatile pump may be actuated to pulsate asynchronously with the patient's heart. Typically, where the patient's heart is beating irregularly, there may be a desire to pulsate the pump 32 asynchronously so that the perfusion of blood by the heart assist system 10 is more regular and, thus, more effective at oxygenating the organs. Where the patient's heart beats regularly, but weakly, synchronous pulsation of the pump 32 may be preferred.

The pump 32 is driven by a motor 40 and/or other type of drive means and is controlled preferably by a programmable controller 42 that is capable of actuating the pump 32 in pulsatile fashion, where desired, and also of controlling the speed or output of the pump 32. For synchronous control, the patient's heart would preferably be monitored with an EKG in which feedback would be provided the controller 42. The controller 42 is preferably programmed by the use of external means. This may be accomplished, for example, using RF telemetry circuits of the type commonly used within implantable pacemakers and defibrillators. The controller may also be autoregulating to permit automatic regulation of the speed, and/or regulation of the synchronous or asynchronous pulsation of the pump 32, based upon feedback from ambient sensors monitoring parameters, such as pressure or the patient's EKG. It is also contemplated that a reverse-direction pump be utilized, if desired, in which the controller is capable of reversing the direction of either the drive means or the impellers of the pump. Such a pump might be used where it is desirable to have the option of reversing the direction of circulation between two blood vessels.

Power to the motor 40 and the controller 42 may be provided by a power source 44, such as a battery, that is preferably rechargeable by an external induction source (not shown), such as an RF induction coil that may be electromagnetically coupled to the battery to induce a charge therein. Alternative power sources are also possible, including a device that draws energy directly from the patient's body; e.g., the patient's muscles, chemicals or heat. The pump can be temporarily stopped during recharging with no appreciable life threatening effect, because the system only supplements the heart, rather than substituting for the heart.

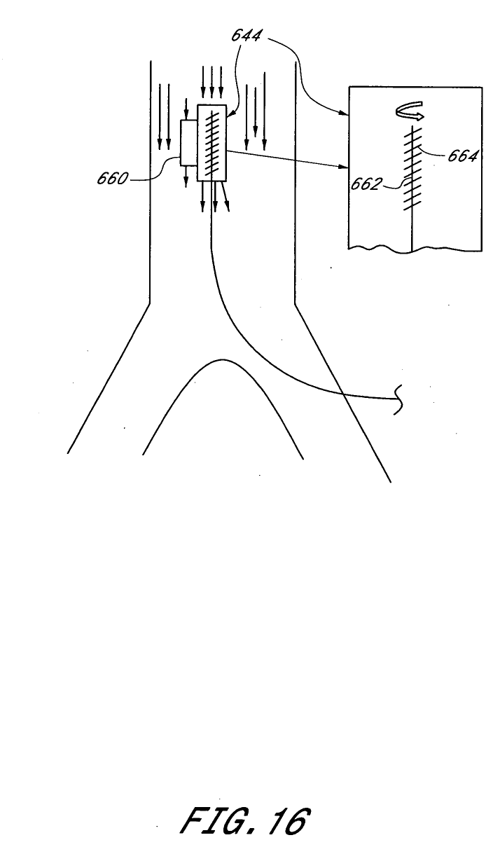

While the controller 42 and power source 44 are preferably pre-assembled to the pump 32 and implanted therewith, it is also contemplated that the pump 32 and motor 40 be implanted at one location and the controller 42 and the power source 44 be implanted in a separate location. In one alternative arrangement, the pump 32 may be driven externally through a percutaneous drive line or cable, as shown in FIG. 16. In another variation, the pump, motor and controller may be implanted and powered by an extracorporeal power source. In the latter case, the power source could be attached to the side of the patient to permit fully ambulatory movement.

The inlet 34 of the pump 32 is preferably connected to an inflow conduit 50 and an outflow conduit 52 to direct blood flow from one peripheral blood vessel to another. The conduits 50, 52 preferably are flexible conduits, as discussed more fully below. The conduits 50, 52 are coupled with the peripheral vessels in different ways in various embodiments of the heart assist system 10. As discussed more fully below, at least one of the conduits 50, 52 can be connected to a peripheral vessel, e.g., as a graft, using an anastomosis connection, and at least one of the conduits 50, 52 can be coupled with the same or another vessel via insertion of a cannula into the vasculature. Also, more than two conduits are used in some embodiments, as discussed below.

The inflow and outflow conduits 50, 52 may be formed from Dacron, Hemashield, Gortex, PVC, polyurethane, PTFE, ePTFE, nylon, or PEBAX materials, although other synthetic materials may be suitable. The inflow and outflow conduits 50, 52 may also comprise biologic materials or pseudobiological (hybrid) materials (e.g., biologic tissue supported on a synthetic scaffold). The inflow and outflow conduits 50, 52 are preferably configured to minimize kinks so blood flow is not meaningfully interrupted by normal movements of the patient or compressed easily from external forces. In some cases, the inflow and/or outflow conduits 50, 52 may come commercially already attached to the pump 32. Where it is desired to implant the pump 32 and the conduits 50, 52, it is preferable that the inner diameter of the conduits 50, 52 be less than 25 mm, although diameters slightly larger may be effective.

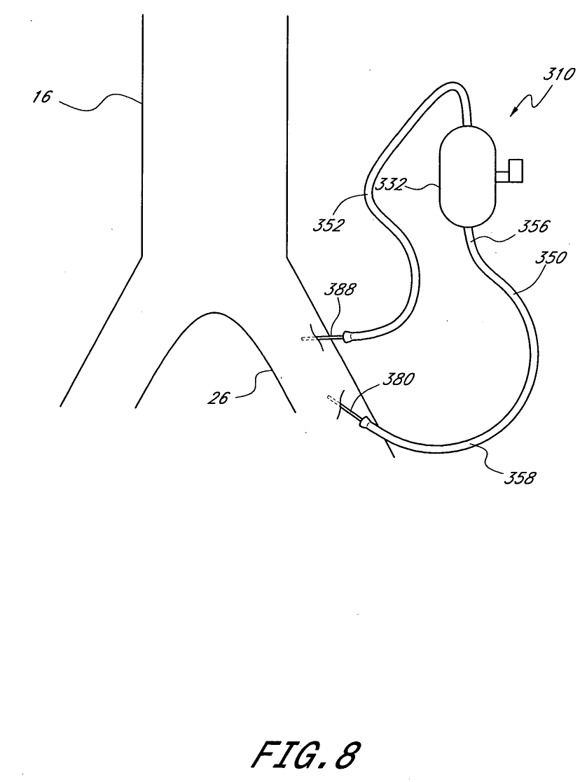

In one preferred application, the heart assist system 10 is applied in an arterial-arterial fashion; for example, as a femoral-axillary connection, as is shown in FIG. 1. It should be appreciated by one of ordinary skill in the art that an axillary-femoral connection would also be effective using the embodiments described herein. Indeed, it should be recognized by one of ordinary skill in the art that the present invention might be applied to any of the peripheral blood vessels in the patient. Another application of the heart assist system 10 couples the conduits 50, 52 with the same non-primary vessel in a manner similar to the application shown in FIG. 8 and discussed below.

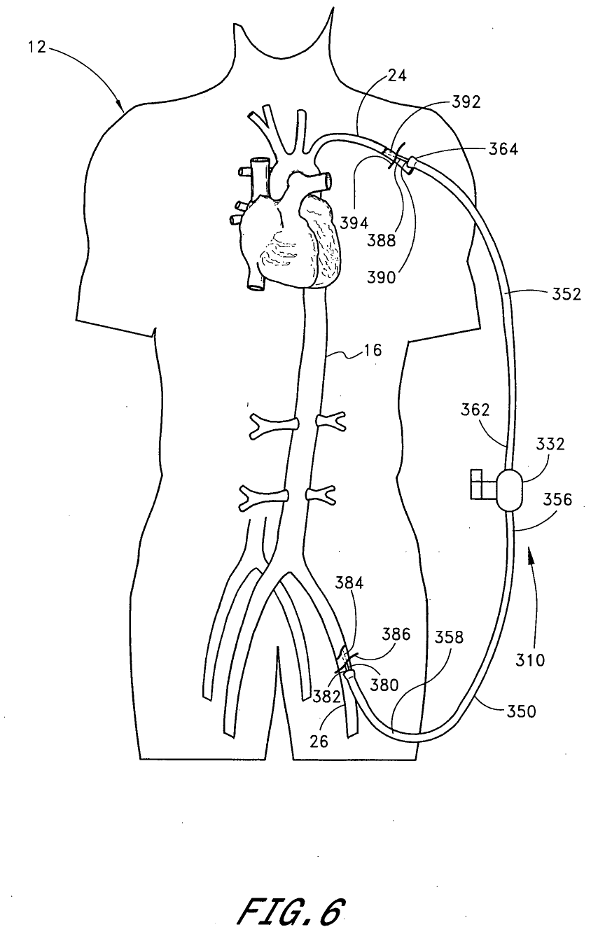

FIG. 1 shows that the inflow conduit 50 has a first end 56 that connects with the inlet 34 of the pump 32 and a second end 58 that is coupled with a first non-primary blood vessel (e.g., the left femoral artery 26) by way of an inflow cannula 60. The inflow cannula 60 has a first end 62 and a second end 64. The first end 62 is sealably connected to the second end 58 of the inflow conduit 50. The second end 64 is inserted into the blood vessel (e.g., the left femoral artery 26). Although shown as discrete structures in FIG. 1, one skilled in the art would recognize that the inflow conduit 50 and the cannula 60 may be unitary in construction. The cannula 60 can take any suitable form, e.g., defining a lumen with an inner size that increases distally as discussed below in connection with FIGS. 17-24.

Where the conduit 50 is at least partially extracorporeal, the inflow cannula 60 also may be inserted through a surgical opening (e.g., as shown in FIG. 6 and described in connection therewith) or percutaneously, with or without an introducer sheath (not shown). In other applications, the inflow cannula 60 could be inserted into the right femoral artery or any other peripheral artery.

FIG. 1 shows that the outflow conduit 52 has a first end 66 that connects to the outlet 36 of the pump 32 and a second end 68 that connects with a second peripheral blood vessel, preferably the left subclavian artery 24 of the patient 12, although the right axillary artery, or any other peripheral artery, would be acceptable. In one application, the connection between the outflow conduit 52 and the second blood vessel is via an end-to-side anastomosis, although a side-to-side anastomosis connection might be used mid-stream of the conduit where the outflow conduit were connected at its second end to yet another blood vessel or at another location on the same blood vessel (neither shown). Preferably, the outflow conduit 52 is attached to the second blood vessel at an angle that results in the predominant flow of blood out of the pump 32 proximally toward the aorta 16 and the heart 14, such as is shown in FIG. 1, while still maintaining sufficient flow distally toward the hand to prevent limb ischemia.

In another embodiment, the inflow conduit 50 is connected to the first blood vessel via an end-to-side anastomosis, rather than via the inflow cannula 60. The inflow conduit 50 could also be coupled with the first blood vessel via a side-to-side anastomosis connection mid-stream of the conduit where the inflow conduit were connected at its second end to an additional blood vessel or at another location on the same blood vessel (neither shown). Further details of these arrangements and other related applications are described in U.S. patent application Ser. No. 10/289,467, filed Nov. 6, 2002, the entire contents of which is hereby incorporated by reference in its entirety and made a part of this specification.

In another embodiment, the outflow conduit 52 also is coupled with the second blood vessel via a cannula, as shown in FIG. 6. This connection may be achieved in a manner similar to that shown in FIG. 1 in connection with the first blood vessel.

It is preferred that application of the heart assist system 10 to the peripheral or non-primary blood vessels be accomplished subcutaneously; e.g., at a shallow depth just below the skin or first muscle layer so as to avoid major invasive surgery. It is also preferred that the heart assist system 10 be applied extrathoracically to avoid the need to invade the patient's chest cavity. Where desired, the entire heart assist system 10 may be implanted within the patient 12, either extravascularly, e.g., as in FIG. 1, or at least partially intravascularly, e.g., as in FIGS. 14-16.

In the case of an extravascular application, the pump 32 may be implanted, for example, into the groin area, with the inflow conduit 50 fluidly connected subcutaneously to, for example, the femoral artery 26 proximate the pump 32. The outflow conduit would be tunneled subcutaneously through to, for example, the left subclavian artery 24. In an alternative arrangement, the pump 32 and associated drive and controller could be temporarily fastened to the exterior skin of the patient, with the inflow and outflow conduits 50, 52 connected percutaneously. In either case, the patient may be ambulatory without restriction of tethered lines.

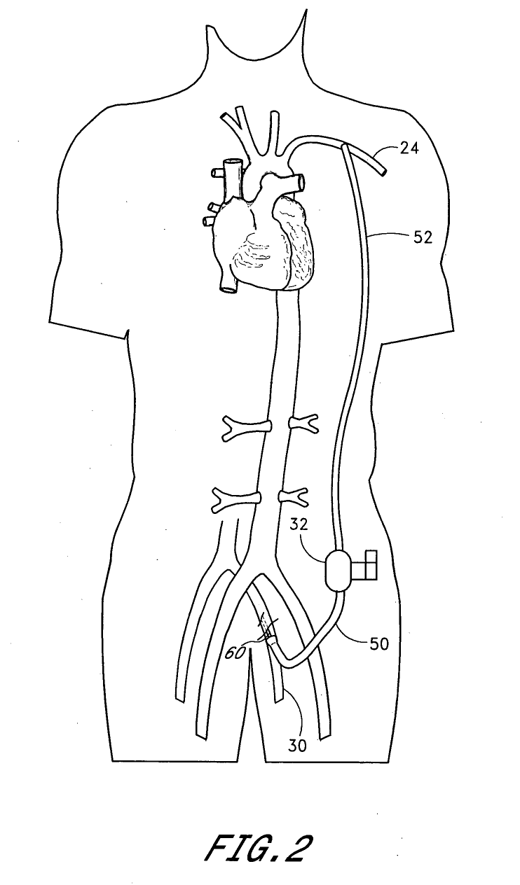

While the heart assist system 10 and other heart assist systems described herein may be applied to create an arterial-arterial flow path, given the nature of the heart assist systems, i.e., supplementation of circulation to meet organ demand, a venous-arterial flow path may also be used. For example, with reference to FIG. 2, one application of the heart assist system 10 couples the inflow conduit 50 with a non-primary vein of the patient 12, such as the left femoral vein 30. In this arrangement, the outflow conduit 50 may be fluidly coupled with one of the peripheral arteries, such as the left subclavian artery 24. Arterial-venous arrangements are contemplated as well. In those venous-arterial cases where the inflow is connected to a vein and the outflow is connected to an artery, the pump 32 should be sized to permit flow sufficiently small so that oxygen-deficient blood does not rise to unacceptable levels in the arteries. It should be appreciated that the connections to the non-primary veins could be by one or more approach described above for connecting to a non-primary artery. It should also be appreciated that the present invention could be applied as a venous-venous flow path, wherein the inflow and outflow are connected to separate peripheral veins. In addition, an alternative embodiment comprises two discrete pumps and conduit arrangements, one being applied as a venous-venous flow path, and the other as an arterial-arterial flow path.

When venous blood is mixed with arterial blood either at the inlet of the pump or the outlet of the pump the ratio of venous blood to arterial blood should be controlled to maintain an arterial saturation of a minimum of 80% at the pump inlet or outlet. Arterial saturation can be measured and/or monitored by pulse oximetry, laser doppler, colorimetry or other methods used to monitor blood oxygen saturation. The venous blood flow into the system can then be controlled by regulating the amount of blood allowed to pass through the conduit from the venous-side connection.

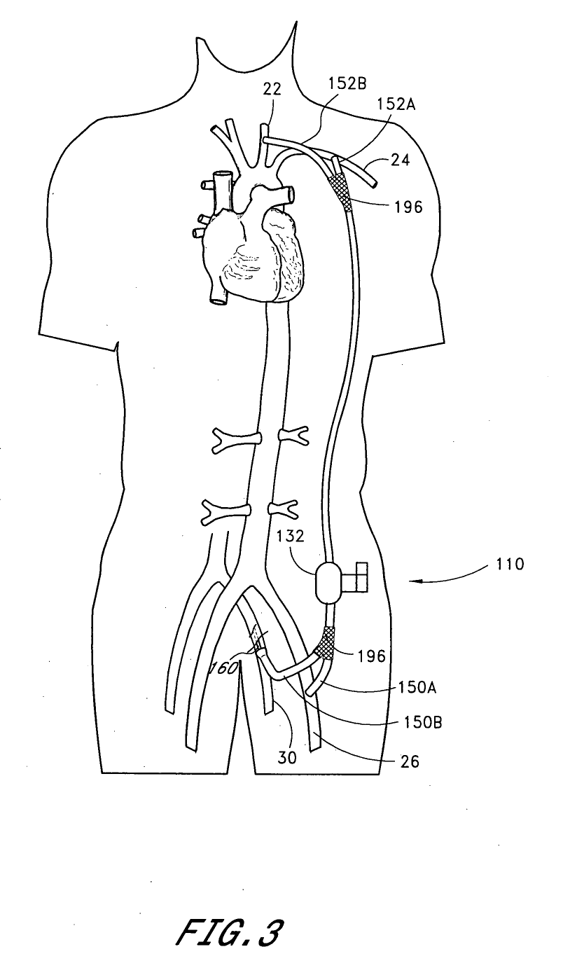

FIG. 3 shows another embodiment of a heart assist system 110 applied to the patient 12. For example, the heart assist system 110 includes a pump 132 in fluid communication with a plurality of inflow conduits 150A, 150B and a plurality of outflow conduits 152A, 152B. Each pair of conduits converges at a generally Y-shaped convergence 196 that converges the flow at the inflow end and diverges the flow at the outflow end. Each conduit may be connected to a separate peripheral blood vessel, although it is possible to have two connections to the same blood vessel at remote locations. In one arrangement, all four conduits are connected to peripheral arteries. In another arrangement, one or more of the conduits could be connected to veins. In the arrangement of FIG. 3, the inflow conduit 150A is connected to the left femoral artery 26 while the inflow conduit 1 SOB is connected to the left femoral vein 30. The outflow conduit 152A is connected to the left subclavian artery 24 while the outflow conduit 152B is connected to the left carotid artery 22. Preferably at least one of the conduits 150A, 150B, 152A, and 152B is coupled with a corresponding vessel via a cannula. In the illustrated embodiment, the inflow conduit 150B is coupled with the left femoral vein 30 via a cannula 160. The cannula 160 is coupled in a manner similar to that shown in FIG. 2 and described in connection with the cannula 60. The cannula 160 can take suitable form, e.g., defining a lumen with an inner size that increases distally as discussed below in connection with FIGS. 17-27.

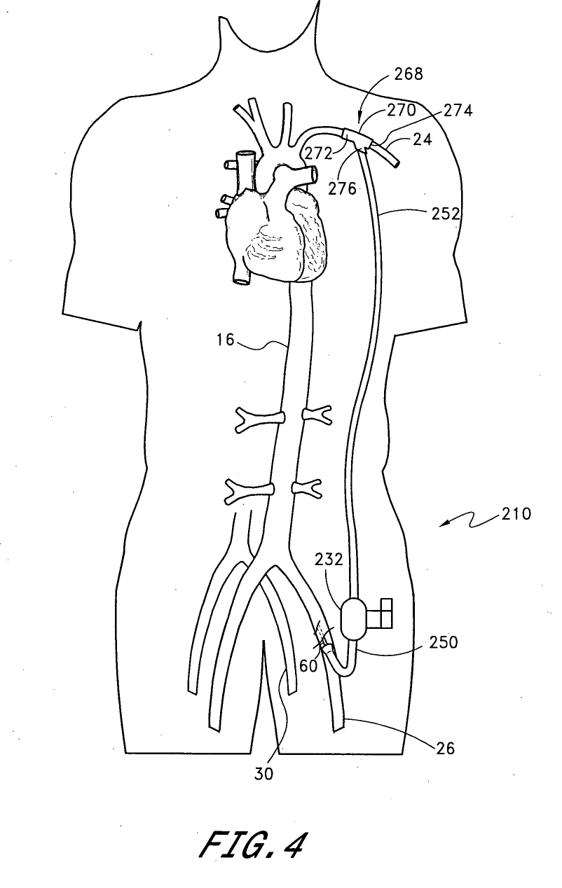

The connections of any or all of the conduits of the system 110 to the blood vessels may be via an anastomosis connection or via a connector, as described below in connection with FIG. 4. In addition, the embodiment of FIG. 3 may be applied to any combination of peripheral blood vessels that would best suit the patient's condition. For example, it may be desired to have one inflow conduit and two outflow conduits or vice versa. It should be noted that more than two conduits may be used on the inflow or outflow side, where the number of inflow conduits is not necessarily equal to the number of outflow conduits.

It is contemplated that, where an anastomosis connection is not desired, a connector may be used to connect at least one of the inflow conduit and the outflow conduit to a peripheral blood vessel. With reference to FIG. 4, an embodiment of a heart assist system 210 is shown, wherein an outflow conduit 252 is connected to a non-primary blood vessel, e.g., the left subclavian artery 24, via a connector 268 that comprises a three-opening fitting. In one embodiment, the connector 268 comprises an intra-vascular, generally T-shaped fitting 270 having a proximal end 272 (relative to the flow of blood in the left axillary artery and therethrough), a distal end 274, and an angled divergence 276 permitting connection to the outflow conduit 252 and the left subclavian artery 24. The proximal and distal ends 274, 276 of the fittings 272 permit connection to the blood vessel into which the fitting is positioned, e.g., the left subclavian artery 24. The angle of divergence 276 of the fittings 272 may be 90 degrees or less in either direction from the axis of flow through the blood vessel, as optimally selected to generate the needed flow distally toward the hand to prevent limb ischemia, and to insure sufficient flow and pressure toward the aorta to provide the circulatory assistance and workload reduction needed while minimizing or avoiding endothelial damage to the blood vessel. In another embodiment, the connector 268 is a sleeve (not shown) that surrounds and attaches to the outside of the non-primary blood vessel where, within the interior of the sleeve, a port to the blood vessel is provided to permit blood flow from the outflow conduit 252 when the conduit 252 is connected to the connector 268.

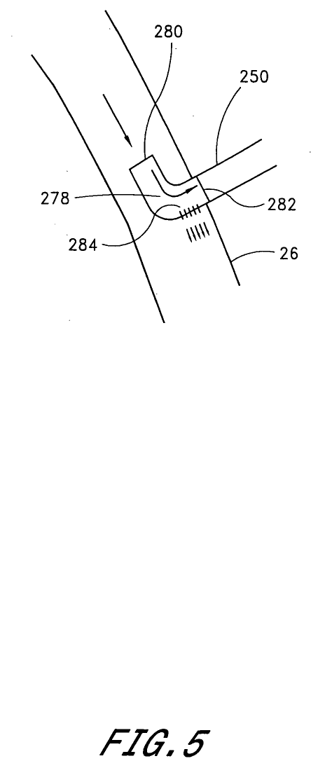

Other types of connectors having other configurations are contemplated that may avoid the need for an anastomosis connection or that permit connection of the conduit(s) to the blood vessel(s). For example, it is contemplated that an L-shaped connector be used if it is desired to withdraw blood more predominantly from one direction of a peripheral vessel or to direct blood more predominantly into a peripheral vessel. Referring to FIG. 5, the inflow conduit 250 is fluidly connected to a peripheral vessel, for example, the left femoral artery 26, using an L-shaped connector 278. Of course the system 210 could be configured so that the outflow conduit 252 is coupled to a non-primary vessel via the L-shaped connector 278 and the inflow conduit 250 is coupled via a cannula, as shown in FIG. 3. The L-shaped connector 278 has an inlet port 280 at a proximal end and an outlet port 282 through which blood flows into the inflow conduit 250. The L-shaped connector 278 also has an arrangement of holes 284 within a wall positioned at a distal end opposite the inlet port 280 so that some of the flow drawn into the L-shaped connector 278 is diverted through the holes 284, particularly downstream of the L-shaped connector 278, as in this application. A single hole 284 in the wall could also be effective, depending upon size and placement. The L-shaped connector 278 may be a deformable L-shaped catheter percutaneously applied to the blood vessel or, in an alternative embodiment, be connected directly to the walls of the blood vessel for more long term application. By directing some blood flow downstream of the L-shaped connector 278 during withdrawal of blood from the vessel, ischemic damage downstream from the connector may be avoided. Such ischemic damage might otherwise occur if the majority of the blood flowing into the L-shaped connector 278 were diverted from the blood vessel into the inflow conduit 252. It is also contemplated that a connection to the blood vessels might be made via a cannula, wherein the cannula is implanted, along with the inflow and outflow conduits.

One advantage of discrete connectors manifests in their application to patients with chronic CHF. A connector eliminates a need for an anastomosis connection between the conduits 250, 252 and the peripheral blood vessels where it is desired to remove and/or replace the system more than one time. The connectors could be applied to the first and second blood vessels semi-permanently, with an end cap applied to the divergence for later quick-connection of the present invention system to the patient. In this regard, a patient might experience the benefit of the heart assist systems described herein periodically, without having to reconnect and redisconnect the conduits 250, 252 from the blood vessels via an anastomosis procedure each time. Each time it is desired to implement any of the embodiments of the heart assist system, the end caps would be removed and a conduit attached to the connector(s) quickly.

In the preferred embodiment of the connector 268, the divergence 276 is oriented at an acute angle significantly less than 90 degrees from the axis of the T-shaped fitting 270, as shown in FIG. 4, so that a majority of the blood flowing through the outflow conduit 252 into the blood vessel (e.g., left subclavian artery 24) flows in a direction proximally toward the heart 14, rather than in the distal direction. In an alternative embodiment, the proximal end 272 of the T-shaped fitting 270 may have a diameter larger than the diameter of the distal end 274, without need of having an angled divergence, to achieve the same result.

With or without a connector, with blood flow directed proximally toward the aorta 16, the result may be concurrent flow down the descending aorta, which will result in the reduction of afterload, impedence, and/or reducing left ventricular end diastolic pressure and volume (preload). Thus, the heart assist systems described herein may be applied so to reduce the afterload on the patient's heart, permitting at least partial if not complete CHF recovery, while supplementing blood circulation. Concurrent flow depends upon the phase of operation of the pulsatile pump and the choice of second blood vessel to which the outflow conduit is connected.

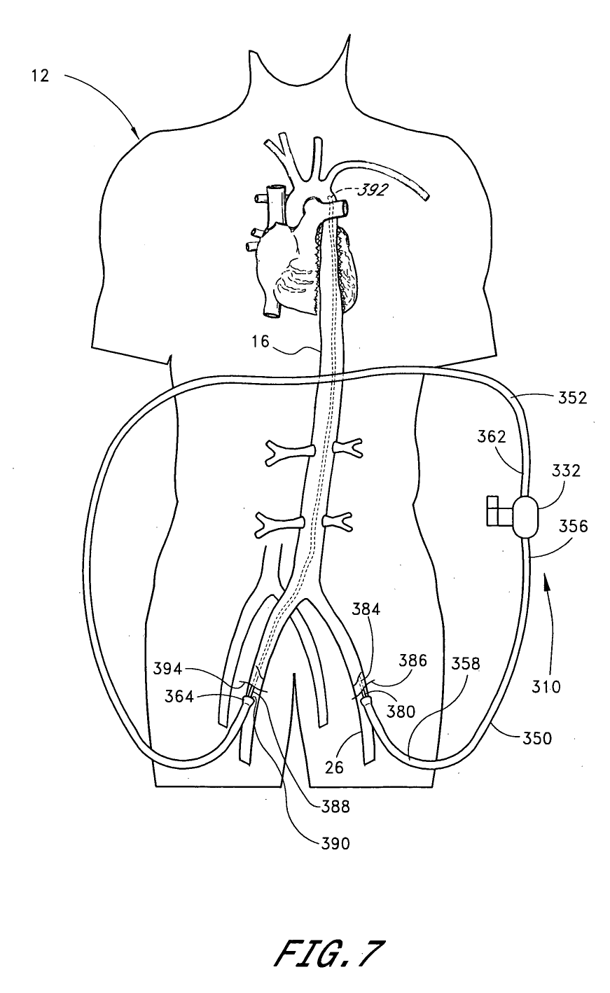

A partial external application of the heart assist systems is contemplated where a patient with heart failure is suffering an acute decompensation episode; i.e., is not expected to last long, or in the earlier stages of heart failure (where the patient is in New York Heart Association Classification (NYHAC) functional classes II or III). With reference to FIGS. 6 and 7, another embodiment of a heart assist system 310 is applied percutaneously to a patient 312 to connect two non-primary blood vessels wherein a pump 332 and its associated driving means and controls are employed extracorporeally. The pump 332 has an inflow conduit 350 and an outflow conduit 352 associated therewith for connection to two non-primary blood vessels. The inflow conduit 350 has a first end 356 and a second end 358 wherein the second end 358 is connected to a first non-primary blood vessel (e.g., femoral artery 26) by way of an inflow cannula 380. The inflow cannula 380 has a first end 382 sealably connected to the second end 358 of the inflow conduit 350. The inflow cannula 380 also has a second end 384 that is inserted through a surgical opening 386 or an introducer sheath (not shown) and into the blood vessel (e.g., the left femoral artery 26).

Similarly, the outflow conduit 352 has a first end 362 and a second end 364 wherein the second end 364 is connected to a second non-primary blood vessel (e.g., the left subclavian artery 24, as shown in FIG. 6, or the right femoral artery 28, as shown in FIG. 7) by way of an outflow cannula 388. Like the inflow cannula 380, the outflow cannula 388 has a first end 390 sealably connected to the second end 364 of the outflow conduit 352. The outflow cannula 388 also has a second end 392 that is inserted through surgical opening 394 or an introducer sheath (not shown) and into the second blood vessel (e.g., the left subclavian artery 24 or the right femoral artery 28). The cannulae 380 and 388 can take any suitable form, e.g., defining a lumen with an inner size that increases distally as discussed below in connection with FIGS. 17-27.

As shown in FIG. 7, the second end 392 of the outflow cannula 388 may extend well into the aorta 16 of the patient 12, for example, proximal to the left subclavian artery. If desired, it may also terminate within the left subclavian artery or the left axillary artery, or in other blood vessels, such as the mesenteric or renal arteries (not shown), where in either case, the outflow cannula 388 has passed through at least a portion of a primary artery (in this case, the aorta 16). Also, if desired, blood drawn into the extracardiac system 310 described herein may originate from the descending aorta (or an artery branching therefrom) and be directed into a blood vessel that is neither the aorta nor pulmonary artery. By use of a percutaneous application, the heart assist system 310 may be applied temporarily without the need to implant any aspect thereof or to make anastomosis connections to the blood vessels.

An alternative variation of the embodiment of FIG. 6 may be used where it is desired to treat a patient periodically, but for short periods of time each occasion and without the use of special connectors. With this variation, it is contemplated that the second ends of the inflow and outflow conduits 350, 352 be more permanently connected to the associated blood vessels via, for example, an anastomosis connection, wherein a portion of each conduit proximate to the blood vessel connection is implanted percutaneously with a removable cap enclosing the externally-exposed first end (or an intervening end thereof) of the conduit external to the patient. When it is desired to provide a circulatory flow path to supplement blood flow, the removable cap on each exposed percutaneously-positioned conduit could be removed and the pump (or the pump with a length of inflow and/or outflow conduit attached thereto) inserted between the exposed percutaneous conduits. In this regard, a patient may experience the benefit of the present invention periodically, without having to reconnect and redisconnect the conduits from the blood vessels each time.

Specific methods of applying this alternative embodiment may further comprise coupling the inflow conduit 352 upstream of the outflow conduit 350 (as shown in FIG. 8), although the reverse arrangement is also contemplated. It is also contemplated that either the cannula 380 coupled with the inflow conduit 350 or the cannula 388 coupled with the outflow conduit 352 may extend through the non-primary blood vessel to a second blood vessel (e.g., through the left femoral artery 26 to the aorta 16 proximate the renal branch) so that blood may be directed from the non-primary blood vessel to the second blood or vice versa.

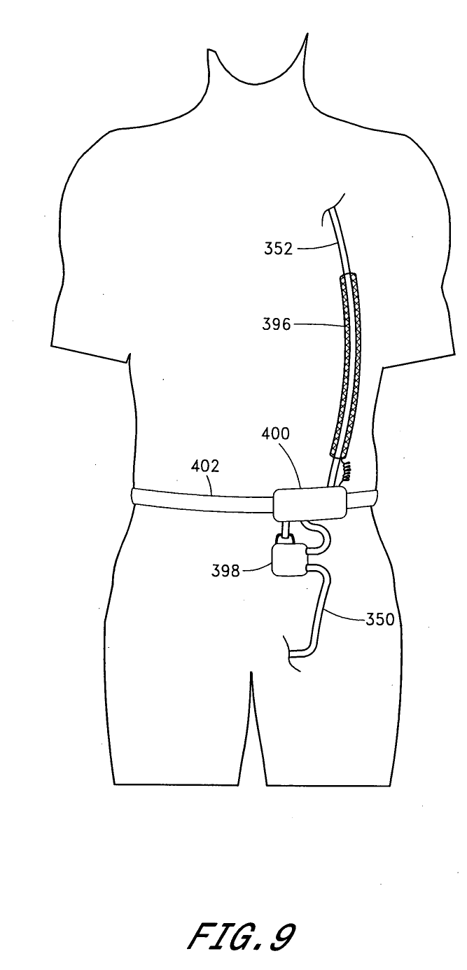

It is contemplated that a means for minimizing the loss of thermal energy in the patient's blood be provided where any of the heart assist systems described herein are applied extracorporeally. Such means for minimizing the loss of thermal energy may comprise, for example, a heated bath through which the inflow and outflow conduits pass or, alternatively, thermal elements secured to the exterior of the inflow and outflow conduits. Referring to FIG. 9, one embodiment comprises an insulating wrap 396 surrounding the outflow conduit 352 having one or more thermal elements passing therethrough. The elements may be powered, for example, by a battery (not shown). One advantage of thermal elements is that the patient may be ambulatory, if desired. Other means that are known by persons of ordinary skill in the art for ensuring that the temperature of the patient's blood remains at acceptable levels while traveling extracorporeally are also contemplated.

If desired, the present inventive system may further comprise a reservoir that is either contained within or in fluid communication with the inflow conduit. This reservoir is preferably made of materials that are nonthrombogenic. Referring to FIG. 9, a reservoir 398 is positioned fluidly in line with the inflow conduit 350. The reservoir 398 serves to sustain adequate blood in the system when the pump demand exceeds momentarily the volume of blood available in the peripheral blood vessel in which the inflow conduit resides until the pump output can be adjusted. The reservoir 398 reduces the risk of excessive drainage of blood from the peripheral blood vessel, which may occur when cardiac output falls farther than the already diminished baseline level of cardiac output, or when there is systemic vasodilation, as can occur, for example, with septic shock. It is contemplated that the reservoir 398 would be primed with an acceptable solution, such as saline, when the present system is first applied to the patient.

As explained above, one of the advantages of several embodiments of the heart assist system is that such systems permit the patient to be ambulatory. If desired, the systems may be designed portably so that it may be carried directly on the patient. Referring to FIG. 9, this may be accomplished through the use of a portable case 400 with a belt strap 402 to house the pump, power supply and/or the controller, along with certain portions of the inflow and/or outflow conduits, if necessary. It may also be accomplished with a shoulder strap or other techniques, such as a backpack or a fanny pack, that permit effective portability As shown in FIG. 9, blood is drawn through the inflow conduit 350 into a pump contained within the portable case 400, where it is discharged into the outflow conduit 352 back into the patient.

B. Heart Assist Systems and Methods Employing Single-Site Application

As discussed above, heart assist systems can be applied to a patient through a single cannulation site. Such single-site systems can be configured with a pump located outside the vasculature of a patient, e.g., as extravascular pumping systems, inside the vasculature of the patient, e.g., as intravascular systems, or a hybrid thereof, e.g., partially inside and partially outside the vasculature of the patient.

1. Single-Site Application of Extravascular Pumping Systems

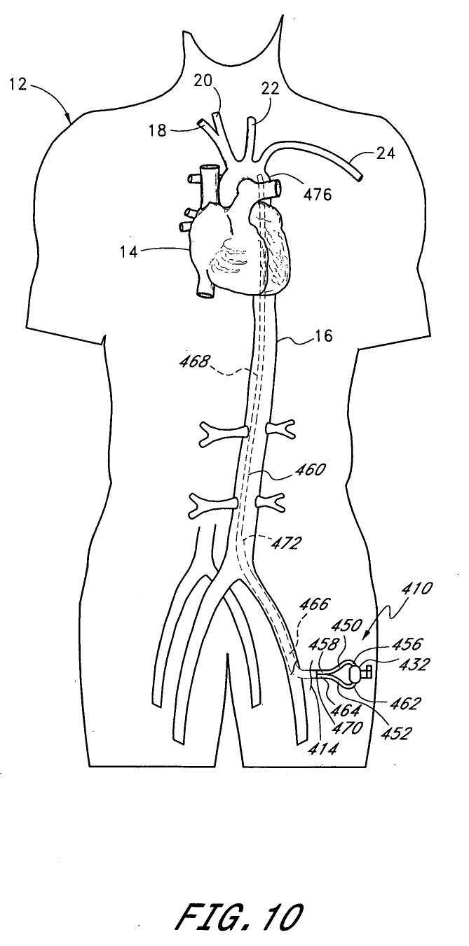

FIGS. 10 and 11 illustrate extracardiac heart assist systems that employ an extravascular pump and that can be applied through as a single-site system. FIG. 10 shows a system 410 that is applied to a patient 12 through a single cannulation site 414 while inflow and outflow conduits fluidly communicate with non-primary vessels. The heart assist system 410 is applied to the patient 12 percutaneously through a single-site to couple two blood vessels with a pump 432. The pump 432 can have any of the features described in connection the pump 32. The pump 432 has an inflow conduit 450 and an outflow conduit 452 associated therewith. The inflow conduit 450 has a first end 456 and a second end 458. The first end 456 of the inflow conduit 450 is connected to the inlet of the pump 432 and the second end 458 of the inflow conduit 450 is fluidly coupled with a first non-primary blood vessel (e.g., the femoral artery 26) by way of a multilumen cannula 460. Similarly, the outflow conduit 452 has a first end 462 and a second end 464. The first end 462 of the outflow conduit 452 is connected to the outlet of the pump 432 and the second end 464 of the outflow conduit 452 is fluidly coupled with a second blood vessel (e.g., the descending aorta 16) by way of the multilumen cannula 460.

In one embodiment, the multilumen cannula 460 includes a first lumen 466 and a second lumen 468. The first lumen 466 extends from a proximal end 470 of the multilumen cannula 460 to a first distal end 472. The second lumen 468 extends from the proximal end 470 to a second distal end 474. In the illustrated embodiment, the second end 458 of the inflow conduit 450 is connected to the first lumen 466 of the multilumen cannula 460 and the second end 464 of the outflow conduit 452 is connected to the second lumen 468 of the multilumen cannula 460.

Where there is a desire for the patient 12 to be ambulatory, the multilumen cannula 460 preferably is made of material sufficiently flexible and resilient to permit the patient 12 to be comfortably move about while the multilumen cannula 460 is indwelling in the patient's blood vessels without causing any vascular trauma.

The application shown in FIG. 10 and described above results in flow from the first distal end 472 to the second distal end 474. Of course, the flow direction may be reversed using the same arrangement, resulting in flow from the distal end 474 to the distal end 472. In some applications, the system 410 is applied in an arterial-arterial fashion. For example, as illustrated, the multilumen cannula 460 can be inserted into the left femoral artery 26 of the patient 12 and guided superiorly through the descending aorta to one of numerous locations. In one application, the multilumen cannula 460 can be advanced until the distal end 474 is located in the aortic arch 476 of the patient 12. The blood could discharge, for example, directly into the descending aorta proximate an arterial branch, such as the left subclavian artery or directly into the peripheral mesenteric artery (not shown).

The pump 432 draws blood from the patient's vascular system in the area near the distal end 472 and into the lumen 466. This blood is further drawn into the lumen of the conduit 450 and into the pump 432. The pump 432 then expels the blood into the lumen of the outflow conduit 452, which carries the blood into the lumen 468 of the multilumen cannula 460 and back into the patient's vascular system in the area near the distal end 474.

FIG. 11 shows another embodiment of a heart assist system 482 that is similar to the heart assist system 410, except as set forth below. The system 482 employs a multilumen cannula 484. In one application, the multilumen cannula 484 is inserted into the left femoral artery 26 and guided superiorly through the descending aorta to one of numerous locations. Preferably, the multilumen cannula 484 has an inflow port 486 that is positioned in one application within the left femoral artery 26 when the cannula 484 is fully inserted so that blood drawn from the left femoral artery 26 is directed through the inflow port 486 into a first lumen 488 in the cannula 484. The inflow port 486 can also be positioned in any other suitable location within the vasculature, described herein or apparent to one skilled in the art. This blood is then pumped through a second lumen 490 in the cannula 484 and out through an outflow port 492 at the distal end of the cannula 484. The outflow port 492 may be situated within, for example, a mesenteric artery 494 such that blood flow results from the left femoral artery 26 to the mesenteric artery 494. The blood could discharge, for example, directly into the descending aorta proximate an arterial branch, such as the renal arteries, the left subclavian artery, or directly into the peripheral mesenteric artery 494, as illustrated in FIG. 11. Where there is a desire for the patient to be ambulatory, the multilumen cannula 484 preferably is made of material sufficiently flexible and resilient to permit the patient 12 to comfortably move about while the cannula 484 is indwelling in the patient's blood vessels without causing any vascular trauma.

Further details of features that may be incorporated into the cannulae, such as the multilumen cannula 460 and the other cannulae described herein are described below in connection with FIGS. 11 and 17-27 and may be found in U.S. patent application Ser. No. 10/078,283, filed Feb. 14, 2002, entitled A MULTILUMEN CATHETER FOR MINIMIZING LIMB ISCHEMIA, U.S. patent application Ser. No. 10/706,346, filed Nov. 12, 2003, entitled CANNULAE HAVING REDIRECTING TIP, U.S. patent application Ser. No. 10/686,040, filed Oct. 15, 2003, entitled IMPLANTABLE HEART ASSIST SYSTEM AND METHOD OF APPLYING SAME, U.S. patent application Ser. No. 10/735,413, filed Dec. 12, 2003, entitled CANNULAE FOR SELECTIVELY ENHANCING BLOOD FLOW, and an application corresponding to Attorneys' Docket ORQIS.021A, entitled CANNULAE HAVING REDUCED FLOW RESISTANCE, filed Jun. 10, 2004, which are hereby expressly incorporated by reference in its entirety and made a part of this specification.

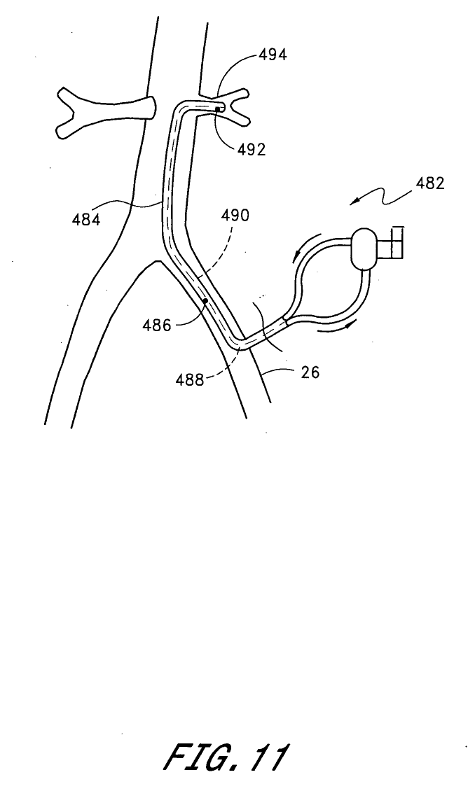

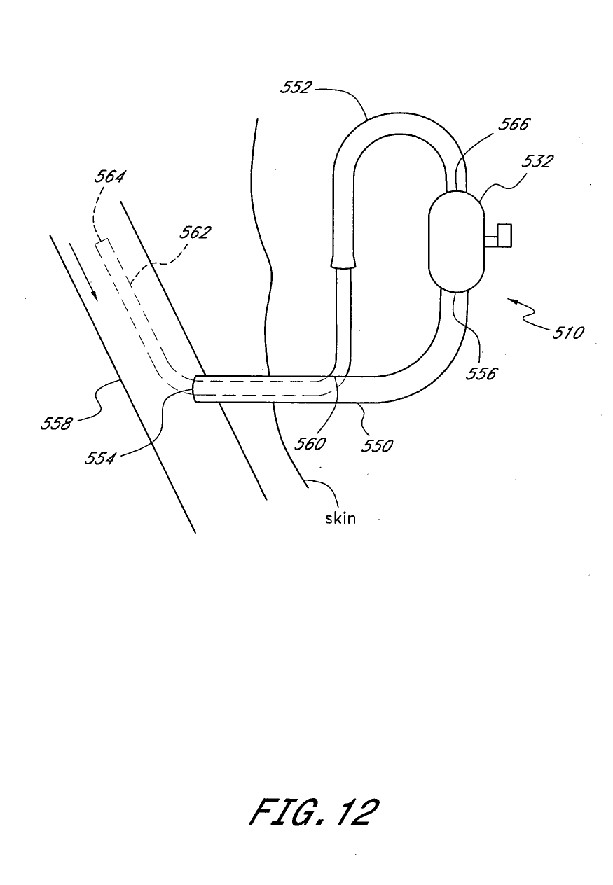

FIG. 12 shows another heart assist system 510 that takes further advantage of the supplemental blood perfusion and heart load reduction benefits while remaining minimally invasive in application. The heart assist system 510 is an extracardiac pumping system that includes a pump 532, an inflow conduit 550 and an outflow conduit 552. In the illustrated embodiment, the inflow conduit 550 comprises a vascular graft. The vascular graft conduit 550 and the outflow conduit 552 are fluidly coupled to pump 532. The pump 532 is configured to pump blood through the patient at subcardiac volumetric rates, and has an average flow rate that, during normal operation thereof, is substantially below that of the patient's heart when healthy. In one variation, the pump 532 may be a rotary pump. Other pumps described herein, or any other suitable pump can also be used in the extracardiac pumping system 510. In one application, the pump 532 is configured so as to be implantable.

The vascular graft 550 has a first end 554 and a second end 556. The first end 554 is sized and configured to couple to a non-primary blood vessel 558 subcutaneously to permit application of the extracardiac pumping system 510 in a minimally-invasive procedure. In one application, the vascular graft conduit 550 is configured to couple to the blood vessel 558 via an anastomosis connection. The second end 556 of the vascular graft 550 is fluidly coupled to the pump 532 to conduct blood between the non-primary blood vessel 558 and the pump 532. In the embodiment shown, the second end 556 is directly connected to the pump 532, but, as discussed above in connection with other embodiments, intervening fluid conducting elements may be interposed between the second end 556 of the vascular graft 550 and the pump 532. Examples of arrangements of vascular graft conduits may be found in U.S. application Ser. No. 09/780,083, filed Feb. 9, 2001, entitled EXTRA-CORPOREAL VASCULAR CONDUIT, which is hereby incorporated by reference in its entirety and made a part of this specification.

FIG. 12 illustrates that the present inventive embodiment further comprises means for coupling the outflow conduit 552 to the vascular graft 550, which may comprise in one embodiment an insertion site 560. In the illustrated embodiment, the insertion site 560 is located between the first end 554 and the second end 556 of the vascular graft 550. The outflow conduit 552 preferably is coupled with a cannula 562. The cannula 562 can take any suitable form, e.g., defining a lumen with an inner size that increases distally as discussed below in connection with FIGS. 17-27.

The insertion site 560 is configured to receive the cannula 562 therethrough in a sealable manner in the illustrated embodiment. In another embodiment, the insertion site 560 is configured to receive the outflow conduit 552 directly. The cannula 562 includes a first end 564 sized and configured to be inserted through the insertion site 560, through the cannula 550, and through the non-primary blood vessel 558. The conduit 552 has a second end 566 fluidly coupled to the pump 532 to conduct blood between the pump 532 and the blood vessel 558.

The extracardiac pumping system 510 can be applied to a patient, as shown in FIG. 12, so that the outflow conduit 552 provides fluid communication between the pump 532 and a location upstream or downstream of the location where the cannula 562 enters the non-primary blood vessel 558. In another application, the cannula 562 is directed through the blood vessel to a different blood vessel, upstream or downstream thereof. Although the vascular graft 550 is described above as an “inflow conduit” and the conduit 552 is described above as an “outflow conduit,” in another application of this embodiment, the blood flow through the pumping system 510 is reversed (i.e., the pump 532 pumps blood in the opposite direction), whereby the vascular graft 550 is an outflow conduit and the conduit 552 is an inflow conduit.

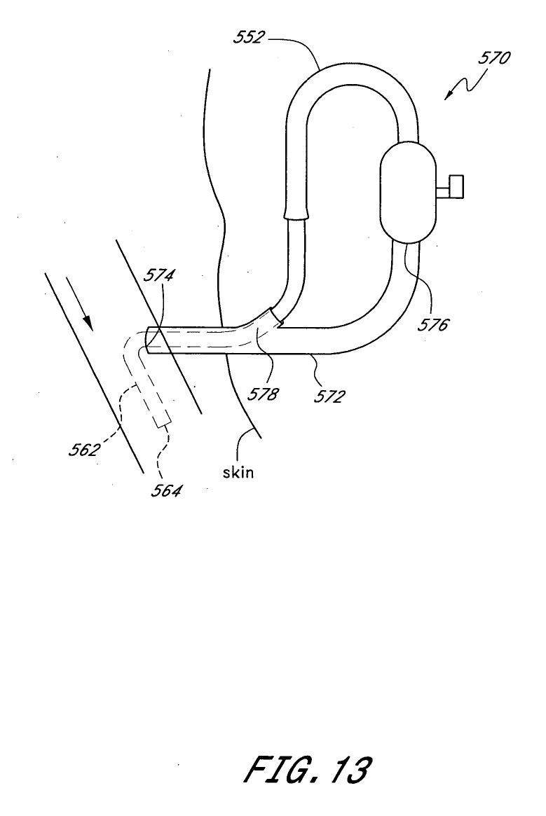

FIG. 13 shows a variation of the extracardiac pumping system shown in FIG. 12. In particular, a heart assist system 570 includes an inflow conduit 572 that comprises a first end 574, a second end 576, and means for connecting the outflow conduit 552 to the inflow conduit 572. In one embodiment, the inflow conduit 572 comprises a vascular graft. The extracardiac pumping system 570 is otherwise similar to the extracardiac pumping system 510. The means for connecting the conduit 552 to the inflow conduit 572 may comprise a branched portion 578. In one embodiment, the branched portion 578 is located between the first end 574 and the second end 576. The branched portion 578 is configured to sealably receive the distal end 564 of the outflow conduit 552. Where, as shown, the first end 564 of the outflow conduit 552 comprises the cannula 562, the branched portion 578 is configured to receive the cannula 562. The inflow conduit 572 of this arrangement comprises in part a multilumen cannula, where the internal lumen extends into the blood vessel 558. Other multilumen catheter arrangements are shown in U.S. application Ser. No. 10/078,283, incorporated by reference herein above.

2. Single-Site Application of Intravascular Pumping Systems

FIG. 14-16 illustrate extracardiac heart assist systems that employ intravascular pumping systems. Such systems take further advantage of the supplemental blood perfusion and heart load reduction benefits discussed above while remaining minimally invasive in application. Specifically, it is contemplated to provide an extracardiac pumping system that comprises a pump that is sized and configured to be at least partially implanted intravascularly in any location desirable to achieve those benefits, while being insertable through a non-primary vessel.

FIG. 14 shows a heart assist system 612 that includes a pumping means 614 comprising preferably one or more rotatable impeller blades 616, although other types of pumping means 614 are contemplated, such as an Archimedes screw, a worm pump, or other means by which blood may be directed axially along the pumping means from a location upstream of an inlet to the pumping means to a location downstream of an outlet from the pumping means. Where one or more impeller blades 616 are used, such as in a rotary pump, such impeller blades 616 may be supported helically or otherwise on a shaft 618 within a housing 620. The housing 620 may be open, as shown, in which the walls of the housing 620 are open to blood flow therethrough. The housing 620 may be entirely closed, if desired, except for an inlet and outlet (not shown) to permit blood flow therethrough in a more channel fashion. For example, the housing 620 could be coupled with or replaced by a cannula with a lumen that has an inner size that increases distally as discussed below in connection with FIGS. 17-27. The heart assist system 612 serves to supplement the kinetic energy of the blood flow through the blood vessel in which the pump is positioned, e.g., the aorta 16.

The impeller blade(s) 616 of the pumping means 614 of this embodiment may be driven in one or a number of ways known to persons of ordinary skill in the art. In the embodiment shown in FIG. 14, the impeller blade(s) 616 are driven mechanically via a rotatable cable or drive wire 622 by driving means 624, the latter of which may be positioned corporeally (intra- or extra-vascularly) or extracorporeally. As shown, the driving means 624 may comprise a motor 626 to which energy is supplied directly via an associated battery or an external power source, in a manner described in more detail herein. It is also contemplated that the impeller blade(s) 616 be driven electromagnetically through an internal or external electromagnetic drive. Preferably, a controller (not shown) is provided in association with this embodiment so that the pumping means 614 may be controlled to operate in a continuous and/or pulsatile fashion, as described herein.

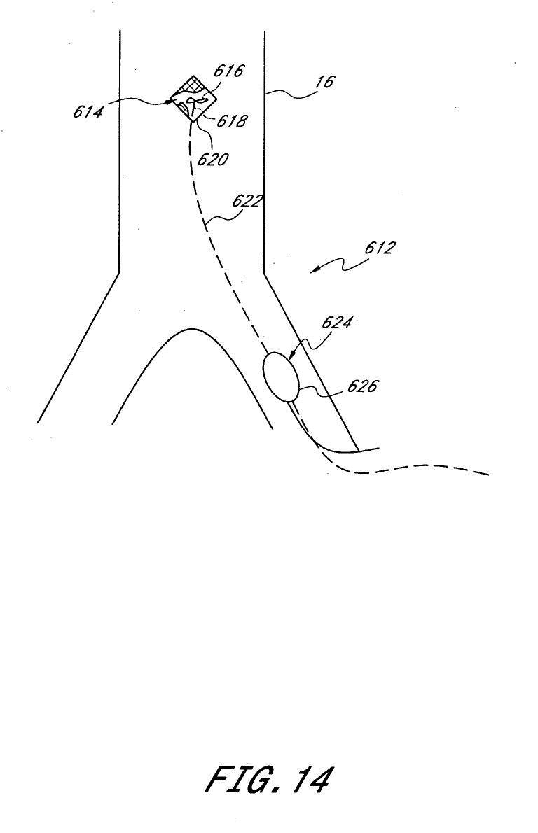

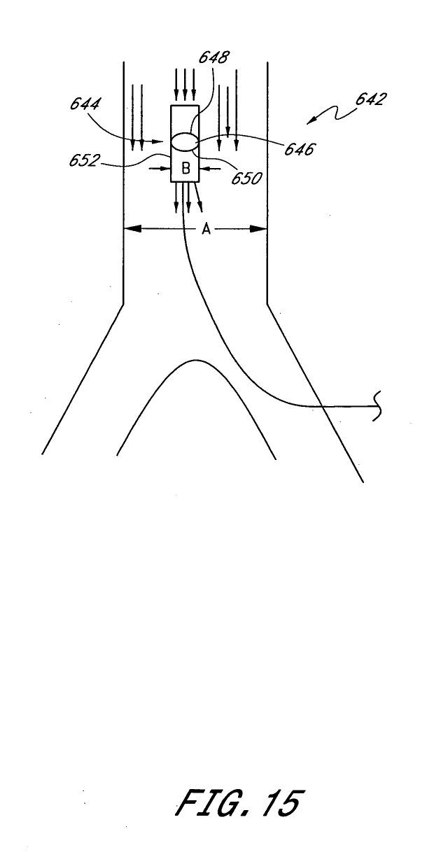

Variations of the intravascular embodiment of FIG. 14 are shown in FIGS. 15 and 16. In the embodiment of FIG. 15, an intrasvascular extracardiac system 642 comprising a pumping means 644, which may be one of several means described herein. The pumping means 644 may be driven in any suitable manner, including means sized and configured to be implantable and, if desired, implantable intravascularly, e.g., as discussed above. For a blood vessel (e.g., descending aorta) having a diameter “A”, the pumping means 644 preferably has a meaningfully smaller diameter “B”. The pumping means 644 may comprise a pump 646 having an inlet 648 and an outlet 650. The pumping means 644 also comprises a pump driven mechanically by a suitable drive arrangement in one embodiment. Although the vertical arrows in FIG. 15 illustrate that the pumping means 644 pumps blood in the same direction as the flow of blood in the vessel, the pumping means 644 could be reversed to pump blood in a direction generally opposite of the flow in the vessel.

In one embodiment, the pumping means 644 also includes a conduit 652 in which the pump 646 is housed. The conduit 652 may be relatively short, as shown, or may extend well within the designated blood vessel or even into an adjoining or remote blood vessel at either the inlet end, the outlet end, or both. The intravascular extracardiac system 642 may further comprise an additional parallel-flow conduit, as discussed below in connection with the system of FIG. 16.

The intrasvascular extracardiac system 642 may further comprise inflow and/or outflow conduits or cannulae (not shown) fluidly connected to the pumping means 644, e.g., to the inlet and outlet of pump 646. Any suitable conduit or cannula can be employed. For example, a cannula defining a lumen with an inner size that increases distally, as discussed below in connection with FIGS. 17-27, could be coupled with an intrasvascular extracardiac system.

In another embodiment, an intrasvascular pumping means 644 may be positioned within one lumen of a multilumen catheter so that, for example, where the catheter is applied at the left femoral artery, a first lumen may extend into the aorta proximate the left subclavian and the pumping means may reside at any point within the first lumen, and the second lumen may extend much shorter just into the left femoral or left iliac. Such a system is described in greater detail in U.S. application Ser. No. 10/078,283, incorporated by reference herein above.

FIG. 16 shows a variation of the heart assist system of FIG. 15. In particular the intravascular system may further comprise an additional conduit 660 positioned preferably proximate the pumping means 644 to provide a defined flow path for blood flow axially parallel to the blood flowing through the pumping means 644. In the case of the pumping means 644 of FIG. 16, the means comprises a rotatable cable 662 having blood directing means 664 supported therein for directing blood axially along the cable. Other types of pumping means are also contemplated, if desired, for use with the additional conduit 660.

The intravascular extracardiac system described herein may be inserted into a patient's vasculature in any means known by one of ordinary skill or obvious variant thereof. In one method of use, such a system is temporarily housed within a catheter that is inserted percutaneously, or by surgical cutdown, into a non-primary blood vessel and advanced through to a desired location. The catheter preferably is then withdrawn away from the system so as not to interfere with operation of the system, but still permit the withdrawal of the system from the patient when desired. Further details of intravascular pumping systems may be found in U.S. patent application Ser. No. 10/686,040, filed Oct. 15, 2003, which is hereby incorporated by reference hereinabove.

C. Potential Enhancement of Systemic Arterial Blood Mixing

One of the advantages of the present invention is its potential to enhance mixing of systemic arterial blood, particularly in the aorta. Such enhanced mixing ensures the delivery of blood with higher oxygen-carrying capacity to organs supplied by arterial side branches off of the aorta. A method of enhancing mixing utilizing the present invention preferably includes taking steps to assess certain parameters of the patient and then to determine the minimum output of the pump that, when combined with the heart output, ensures turbulent flow in the aorta, thereby enhancing blood mixing.

Blood flow in the aortic arch during normal cardiac output may be characterized as turbulent in the end systolic phase. It is known that turbulence in a flow of fluid through pipes and vessels enhances the uniform distribution of particles within the fluid. It is believed that turbulence in the descending aorta enhances the homogeneity of blood cell distribution in the aorta. It is also known that laminar flow of viscous fluids leads to a higher concentration of particulate in the central portion of pipes and vessels through which the fluid flows. It is believed that, in low flow states such as that experienced during heart failure, there is reduced or inadequate mixing of blood cells leading to a lower concentration of nutrients at the branches of the aorta to peripheral organs and tissues. As a result, the blood flowing into branch arteries off of the aorta will likely have a lower hematocrit, especially that flowing into the renal arteries, the celiac trunk, the spinal arteries, and the superior and inferior mesenteric arteries. That is because these branches draw from the periphery of the aorta The net effect of this phenomenon is that the blood flowing into these branch arteries has a lower oxygen-carrying capacity, because oxygen-carrying capacity is directly proportional to both hematocrit and the fractional O₂ saturation of hemoglobin. Under those circumstances, it is very possible that these organs will experience ischemia-related pathology.

The phenomenon of blood streaming in the aorta, and the resultant inadequate mixing of blood resulting in central lumenal concentration of blood cells, is believed to occur when the Reynolds number (NR) for the blood flow in the aorta is below 2300. To help ensure that adequate mixing of blood will occur in the aorta to prevent blood cells from concentrating in the center of the lumen, a method of applying the present invention to a patient may also include steps to adjust the output of the pump to attain turbulent flow within the descending aorta upstream of the organ branches; i.e., flow exhibiting a peak Reynolds number of at least 2300 within a complete cycle of systole and diastole. Because flow through a patient is pulsatile in nature, and not continuous, consideration must be given to how frequently the blood flow through the aorta has reached a certain desired velocity and, thus, a desired Reynolds number. The method contemplated herein, therefore, should also include the step of calculating the average Womersley number (N_{w}), which is a function of the frequency of the patient's heart beat. It is desired that a peak Reynolds number of at least 2300 is attained when the corresponding Womersley number for the same blood flow is approximately 6 or above.

More specifically, the method may comprise calculating the Reynolds number for the blood flow in the descending aorta by determining the blood vessel diameter and both the velocity and viscosity of the fluid flowing through the aorta. The Reynolds number may be calculated pursuant to the following equation:

$${N}_{R} = \frac{V \cdot d}{υ}$$

where: V=the velocity of the fluid; d=the diameter of the vessel; and υ=the viscosity of the fluid. The velocity of the blood flowing through the aorta is a function of the cross-sectional area of the aorta and the volume of flow therethrough, the latter of which is contributed both by the patient's own cardiac output and by the output of the pump of the present invention. Velocity may be calculated by the following equation:

$$V = \frac{Q}{π {r}^{2}}$$
where Q=the volume of blood flowing through the blood vessel per unit time, e.g., the aorta, and r=radius of the aorta. If the relationship between the pump output and the velocity is already known or independently determinable, the volume of blood flow Q may consist only of the patient's cardiac output, with the knowledge that that output will be supplemented by the subcardiac pump that is part of the present invention. If desired, however, the present system can be implemented and applied to the patient first, before calculating Q, which would consist of the combination of cardiac output and the pump output.

The Womersley number may be calculated as follows:
N_{w}=\sqrt{2πω/υ}
where r is the radius of the vessel being assessed, ω is the frequency of the patient's heartbeat, and υ=the viscosity of the fluid. For a peak Reynolds number of at least 2300, a Womersley number of at least 6 is preferred, although a value as low as 5 would be acceptable.

By determining (i) the viscosity of the patient's blood, which is normally about 3.0 mm²/sec (kinematic viscosity), (ii) the cardiac output of the patient, which of course varies depending upon the level of CHF and activity, and (iii) the diameter of the patient's descending aorta, which varies from patient to patient but is about 21 mm for an average adult, one can determine the flow rate Q that would result in a velocity through the aorta necessary to attain a Reynolds number of at least 2300 at its peak during the patient's heart cycle. Based upon that determination of Q, one may adjust the output of the pump of the present invention to attain the desired turbulent flow characteristic through the aorta, enhancing mixing of the blood therethrough.

One may use ultrasound (e.g., echocardiography or abdominal ultrasound) to measure the diameter of the aorta, which is relatively uniform in diameter from its root to the abdominal portion of the descending aorta. Furthermore, one may measure cardiac output using a thermodilution catheter or other techniques known to those of skill in the art. Finally, one may measure viscosity of the patient's blood by using known methods; for example, using a capillary viscosimeter. It is expected that in many cases, the application of this embodiment of the present method will provide a basis to more finely tune the system to more optimally operate the system to the patient's benefit. Other methods contemplated by the present invention may include steps to assess other patient parameters that enable a person of ordinary skill in the art to optimize the present system to ensure adequate mixing within the vascular system of the patient.

Alternative inventive methods that provide the benefits discussed herein include the steps of, prior to applying a shape change therapy, applying a blood supplementation system (such as one of the many examples described herein) to a patient, whereby the methods are designed to improve the ability to reduce the size and/or wall stress of the left ventricle, or both ventricles, thus reducing ventricular loading. Specifically, one example of such a method comprises the steps of providing a pump configured to pump blood at subcardiac rates, providing inflow and outflow conduits configured to fluidly communicate with non-primary blood vessels, fluidly coupling the inflow conduit to a non-primary blood vessel, fluidly coupling the outflow conduit to the same or different (primary or non-primary) blood vessel and operating the subcardiac pump in a manner, as described herein, to reduce the load on the heart, wherein the fluidly coupling steps may comprise anastomosis, percutaneous canalization, positioning the distal end of one or both conduits within the desired terminal blood vessel or any combination thereof. The method further comprises, after sufficient reduction in ventricular loading, applying a shape change therapy in the form of, for example, a cardiac reshaping device, such as those referred to herein, or others serving the same or similar function, for the purpose of further reducing the size of and/or wall stress on one or more ventricles and, thus, the heart, and/or for the purpose of maintaining the patient's heart at a size sufficient to enhance recovery of the patient's heart.

II. Cannulae for Use in Extracardiac Heart Assist Systems

As discussed above, application of a heart assist system to a patient can involve inserting a cannula into the patient's vasculature to deliver and/or withdraw blood. Such cannulae may be single lumen (see, e.g., FIGS. 1-9 and 12-13) or multilumen (see, e.g., FIGS. 10-11 and FIGS. 17-27). The cannulae discussed below are shown as multilumen cannulae and thus are particularly well suited for single-site applications. The embodiments discussed below may be modified according to or used with any of the single-site apparatuses, systems, or methods discussed above. The embodiments discussed below can also be combined with each other in any suitable manner to provide apparatuses, systems, or methods useful in other contexts. Also, the embodiments discussed below can be used in apparatuses, systems, and methods directed at multi-site applications or treatments. In some embodiments, the cannulae provide variable lumen size, e.g., increasing size toward the distal end of the cannulae. A variety of features and combinations that facilitate insertion of the cannulae also are described below.

A. Multilumen Cannulae

Referring to FIGS. 17-18C, one embodiment of a multilumen cannula 666 that includes a first elongate portion 668 defining a first lumen 670 and a second elongate portion 672 defining a second lumen 674. The first elongate portion 668 extends between a first distal end 676 and a proximal end 680. The second elongate portion 672 extends between a second distal end 678 and the proximal end 680. The first distal end 676 of the first elongate portion 668 extends distally farther from the proximal end 680 of the multilumen cannula 666 than does the second distal end 678.

The multilumen cannula 666 includes a proximal portion 682 wherein the first and second elongate portions 668, 672 extend generally side-by-side, at least partially separated by a wall 684. As shown in FIG. 18C, the elongate portions 668, 672 and the wall 684 in the proximal portion 682 of the multilumen cannula 666 form two lumens with D-shaped cross-sections 670, 674. Although the lumens 670, 674 are shown as having approximately the same size, other relative sizes are possible. For example, the shorter lumen 674 could be made smaller than the longer lumen 670. Other arrangements of side-by-side lumens are also possible, e.g., where the lumens have shapes other than that shown in FIG. 18C. For example, the lumens 670, 674 could be circular in cross-section (or any other suitable shape) rather than D-shaped.

With reference to FIGS. 18A and 18B, the inner cross-sectional size of the first lumen 670 expands distal the second distal end 676 compared to the inner cross-sectional size of the first lumen 670 in the proximal portion 682 of the multilumen cannula 666. The expanded size of the first lumen 670 makes the inner cross-sectional area of the first lumen 670 greater at the first distal end 676 than at the proximal end 680. In one embodiment, the elongate portion 668 of the multilumen cannula 666 increases from about a seven French size in the proximal portion 682 to about a twelve French size in the distal portion 692. In other embodiments, at least about a one hundred percent increase in the size of the lumen 670 in the elongate portion 668 at the distal end 676 compared to the proximal end 680 is provided. The length of the transition portion 690 may be any suitable length, e.g., one that provides gradual increase distally to prevent abrupt changes in aspects of the flow of the blood (e.g., the flow direction). In one embodiment, the length of the transition portion 690 is about one inch. In one embodiment, the length of the transition portion 690 is about one inch or less. In another embodiment, the length of the transition portion 690 is about one-half inch. As previously discussed, increasing the inner cross-section size of the first lumen 670 at any point along the length of the cannula 666 will decrease the overall flow resistance of a heart-assist system employing the cannula 666. It is expected that the decrease in flow resistance would be most significant when the inner cross-section of the first lumen 670 is increased for as much of the length as is possible.

The cannula 666 has a transition portion 690 wherein the cross sectional size of the first elongate portion 668 expands. The transition portion 690 preferably extends from proximate the second distal end 678 of the second elongate portion 672 to a location 688 distal the second distal end 678. The cross-sectional size of the cannula 666 distal the location 688 preferably is about equal to the cross-sectional size of the proximal portion 682 at a location 686 just proximal the second distal end 678.

With reference to FIG. 18B, the cross-section size of the lumen 670 increases from proximal to distal within the transition portion 690. The increase in cross-section size of the lumen 670 may be achieved in any suitable manner. Preferably, the location of the wall 684 in the transition portion 690 gradually moves transversely from proximal to distal such that the D-shape of the lumen 670 in the proximal portion 682 of the cannula 666 transitions gradually to a more circular cross-sectional shape toward the distal end of the transition portion 690. Distal the location 688 (e.g., at a location 694) the inner cross-sectional shape of the first lumen 670 preferably becomes circular, as illustrated in FIG. 18A. The cross-sectional size of the lumens 670, 674 (or the lumens of any of the other cannulae described herein) may vary from proximal to distal in at least one of the proximal portion 682 and the distal portion 692.

The cannula 666 preferably comprises a distal portion 692 wherein the cross-sectional size of the cannula 666 is substantially the same as the cross-sectional size of the cannula 666 in the proximal portion 682, and the interior cross-section of the first lumen 670 is circular.

The multilumen cannula 666 is also configured in an advantageous manner for insertion into the vasculature of a patient. The proximal and distal portions 682, 692 of the multilumen cannula 666 provide a substantially constant outer cross-sectional profile. In particular, the outer cross-sectional size of the multilumen cannula 666 is substantially the same at the location 686, immediately proximal the second distal end 678 and at the location 688, immediately distal the transition portion 690.

In some embodiments, it may be desirable to minimize the length of the transition portion 690 to ease insertion of the cannula 666 into the vasculature of a patient. Minimizing the transition portion 690 is further advantageous because the length of the distal, portion 692 may be increased to further reduce the overall flow resistance of the cannula 666. However, factors such as the amount of blood flow through the second distal end 678 and the flow of blood through the lumen 670 within the transition portion 690 may place a lower limit on the length of the transition portion 690.

In order to minimize the flow resistance in the cannula 666, it is desirable to design the cannula so that the distal portion 692 comprises as much of the total length of the cannula 666 as is possible, given other constraints on the cannula 666. Thus, the length of the proximal portion 682, and therefore the length of the second lumen 674, will be minimized as much as is possible. The flow resistance will thus be decreased both because the portion of the first lumen 670 that is increased in size is increased and the portion of the first lumen 670 that is decrease in size is decreased.

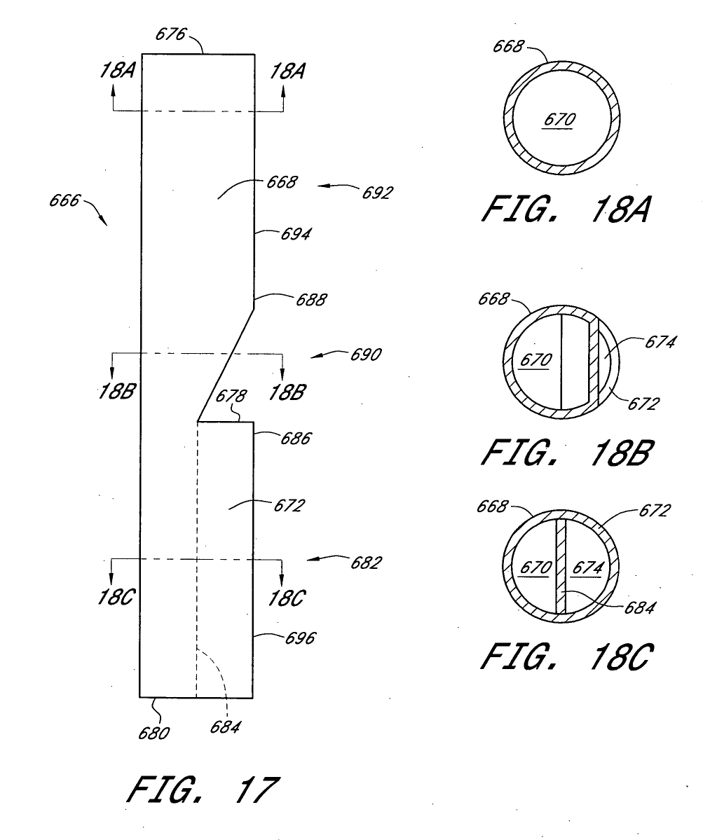

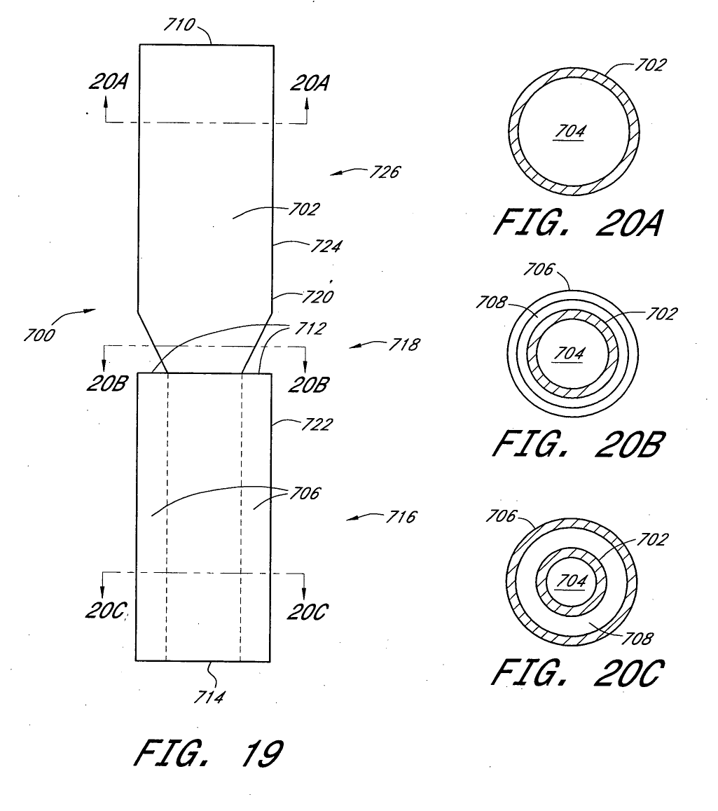

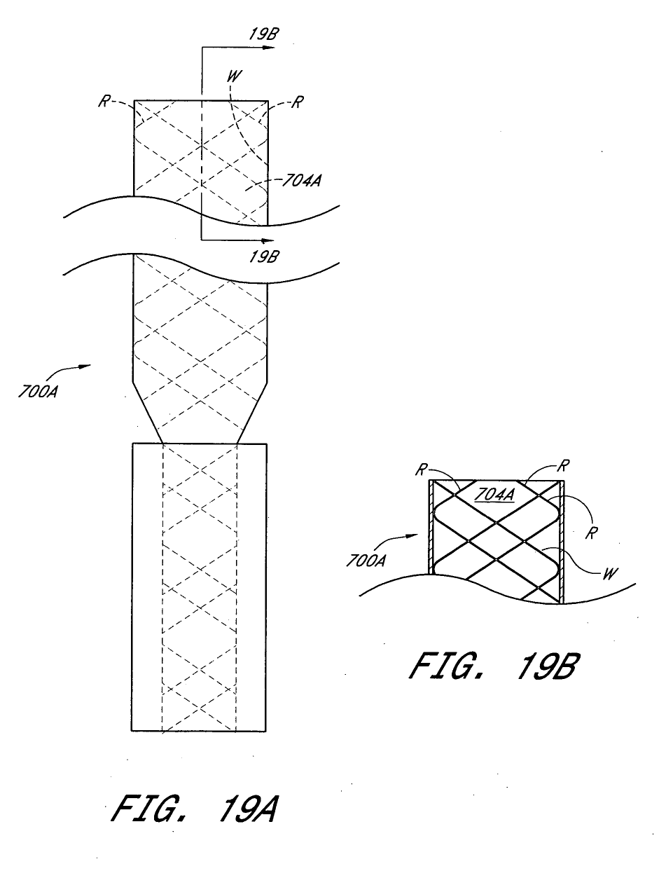

Referring to FIG. 19, another embodiment of a multilumen cannula 700 includes a first elongate portion 702 defining a first lumen 704 and a second elongate portion 706 defining a second lumen 708. The lumens 704, 708 are shown more clearly in FIGS. 20A-20C. The first elongate portion 702 extends between a first distal end 710 and a proximal end 714. The second elongate portion 706 extends between a second distal end 712 and the proximal end 714. The first distal end 710 of the first elongate portion 702 extends distally farther from the proximal end 714 of the multilumen cannula 700 than does the second distal end 712.

In this embodiment, the multilumen cannula 700 includes a proximal portion 716, a transition portion 718, and a distal portion 726. In the proximal portion 716 of the cannula 700, the first and second elongate portions 702, 706 extend generally parallel to each other. In the illustrated embodiment, the first elongate portion 702 extends through the second lumen 708 defined in the second elongate portion 706. In this arrangement, the first and second elongate portions 702, 706 form two concentric circles in cross-section, as shown in FIGS. 20B-20C.

The transition portion 718 of the multilumen cannula 700 preferably extends from a location proximate to the second distal end 712 of the second elongate portion 706 to a location 720 longitudinally between the second distal end 712 and the first distal end 710. The first elongate portion 702 generally expands distally in the transition portion 718. In one embodiment, the transition portion 718 expands distally continuously. In another embodiment, the transition portion 718 expands distally continuously and at a constant rate. The expansion of the first elongate portion 702 corresponds to an increase in the girth of the elongate portion 702, e.g., to an increase in the outer diameter thereof. In one embodiment, the thickness of the wall defining the elongate portion 702 is held constant from proximal to distal through the transition portion 718. Because the wall thickness is constant, and the outer size of the elongate portion 702 in the transition portion 718 is expanding, the first lumen 704 in the transition portion correspondingly increases from proximal to distal. In one embodiment, the elongate portion 702 increases from about a seven French size in the proximal portion 716 to about a twelve French size in the distal portion 726. In other embodiments, at least about a one hundred percent increase in the size of the lumen 704 in the elongate portion 702 at the distal end 712 compared to the proximal end 714 is provided. The length of the transition portion 718 may be any suitable length, e.g., one that provides gradual increase distally to prevent abrupt changes in aspects of the flow direction of the blood (e.g., the flow direction). In one embodiment, the length of the transition portion 718 is about one-half inch. In one embodiment, the length of the transition portion 718 is about one inch or less. In another embodiment, the length of the transition portion 718 is about one inch. As discussed above, this increase advantageously increases the cross-sectional area of the lumen through which blood may flow, which reduces the magnitude of fluid-dynamic losses due to flow resistance. Of course, the thickness of the wall defining the elongate portion 702 in the transition portion 718 need not remain constant. Rather the wall can thicken or become thinner as desired.

In one embodiment, the cross-section shape of the first lumen 704 in the transition portion 718 is the same as the cross-sectional shape of the first lumen 704 in the proximal portion 716. In one embodiment, the cross-sectional shape of the first lumen 704 in the transition portion 718 and in the proximal portion 716 is circular.

The distal portion 726 of the cannula 700 is that portion residing distal the transition portion 718. The size of the distal portion 726 of the cannula 700 (e.g., the outer diameter) preferably is substantially the same as the size of the proximal portion 716 of the cannula 700. The shape of the second lumen 704 in the distal portion 726 preferably is the same as the shape of the second lumen 704 in the transition portion 718, e.g., circular. The circular cross-sectional shape of the second lumen 704 in the distal portion 726 is shown in FIG. 20A.

With reference to FIGS. 20A and 20B, the inner cross-sectional size of the first lumen 704 expands distal the second distal end 712 compared to the inner cross-sectional size of the first lumen 704 in the proximal portion 716 of the multilumen cannula 700. The expanded size of the first lumen 704 makes the inner cross-section of the first lumen 704 greater at the first distal end 710 than at the proximal end 714. As previously discussed, this configuration is advantageous in that the cannula 700 has lower flow resistance compared to a cannula of comparable length with a constant inner cross-sectional size equal to inner cross-sectional size of the lumen 704 in the proximal portion 716 of the cannula 700.

The multilumen cannula 700 is also configured in an advantageous manner for insertion into the vasculature of a patient. In the illustrated embodiment, both the proximal portion 716 and the distal portion 726 provide a substantially constant outer cross-sectional profile. In particular, the outer cross-sectional size of the multilumen cannula 700 is substantially the same at a location 722 immediately proximal the second distal end 712 and at a location 720 immediately distal the transition portion 718.

As discussed above in connection with FIG. 17, minimizing the length of the transition portion 718 may be advantageous. Also, it is desirable for the distal portion 724 of the first elongate portion 702 to be as long as possible and for the proximal portion 716 of the first elongate portion 702 to be as short as possible, given other constraints on the cannula design.

Referring to FIG. 19A, another embodiment of a multilumen cannula 700A is configured to impart a rotational component to the flow of fluid therein (e.g., a vortex flow). The cannula 700A is similar to the cannula 700, except as set forth below. In one embodiment, the walls W that surround a lumen 704A of the cannula 700A are configured to impart a rotation component to the flow of fluid in the lumen 704A. Any suitable structure may be employed to impart the rotational component to the flow. One benefit of imparting a rotational component to the flow is that resistance to flow may be reduced, providing some or all of the benefits of reduced resistance flow, including those described herein.

In one embodiment, the walls W of the cannula 700A is configured to impart a rotational component of the flow of fluid therein. In one embodiment, the walls W of the cannula 700A are provided with at least one ridge R formed thereon. Any suitable configuration of the ridge R may be employed. The ridge R may be arcuate, spiraled, helical, or any other suitable shape that will impart a rotational component to the flow. In the spiraled embodiment, the density of the spiral may be any suitable density. For example, the spiral ridge R may extend about once around (e.g., about 360 degrees around) the lumen 704A of the cannula 700A per inch of length of the cannula 700A. In another embodiment, the spiral ridge R may extend as many as about ten times around the lumen 704A of the cannula 700A per inch of length, or more. In another embodiment, the spiral ridge R may extend about once around the lumen 704A per ten inches of the cannula 700A, or less.

In the illustrated embodiment, a plurality of ridges R is provided. In particular, with reference to FIG. 19A, four ridges R are provided in the lumen 704A. Other numbers of ridges may also be provided to create vortex flow, e.g., more than four, three, two, or one ridge may be provided. In one embodiment, a plurality of ridges R is provided wherein the ridges R are off-set from each other about the circumference of the lumen 704A. For example, two ridges R may be located directly across the lumen 704A from each other (e.g., spaced 180 degrees apart). In one embodiment, at least one of the ridges R extends from the proximal end to the distal end of the lumen 704A. In another embodiment, at least one of the ridges R extends less than the entire length of the lumen 704A. The ridges R and the internal structure of a portion of the lumen 704A of the cannula 700A are shown in greater detail in FIG. 19B.

As discussed herein, providing a cannula with a lumen that transitions to a larger size in at least a portion of a distal portion compared with a proximal portion can reduce flow resistance in the lumen compared to non-distally increasing lumen cannula. Configuring the lumen 704A to impart a rotational component to the flow of fluid therein similarly reduces the resistance to the flow of fluid in the lumen 704A. The cannula 700A combines the benefits of increased lumen size, as discussed above in connection with the cannula 700, with the benefits of providing the ridge(s) R. In some embodiments, the cannula 700A may be have one or more ridges R as shown in FIG. 19A, but not have an increased lumen size. Such an arrangement can provide advantageous flow resistance reduction in some applications.

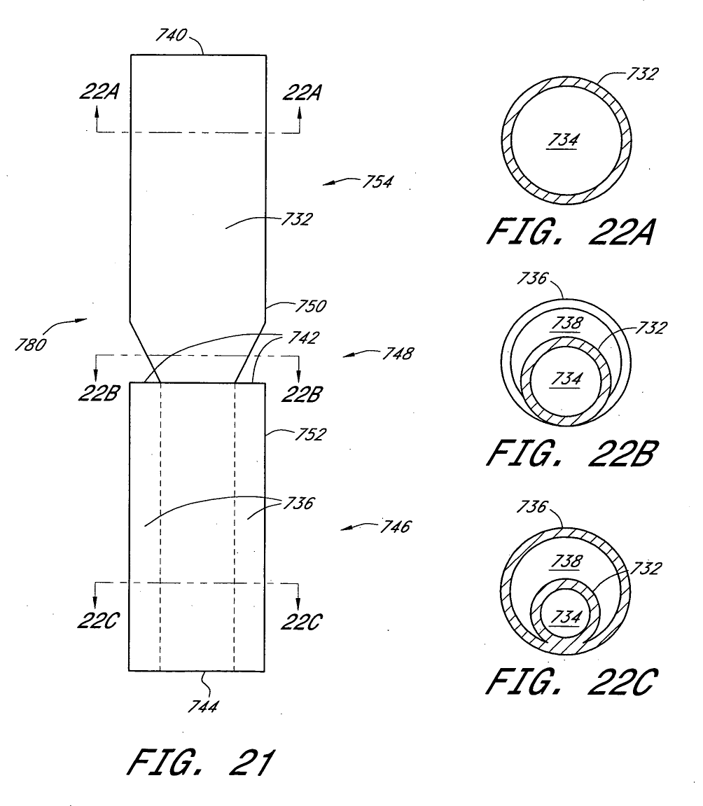

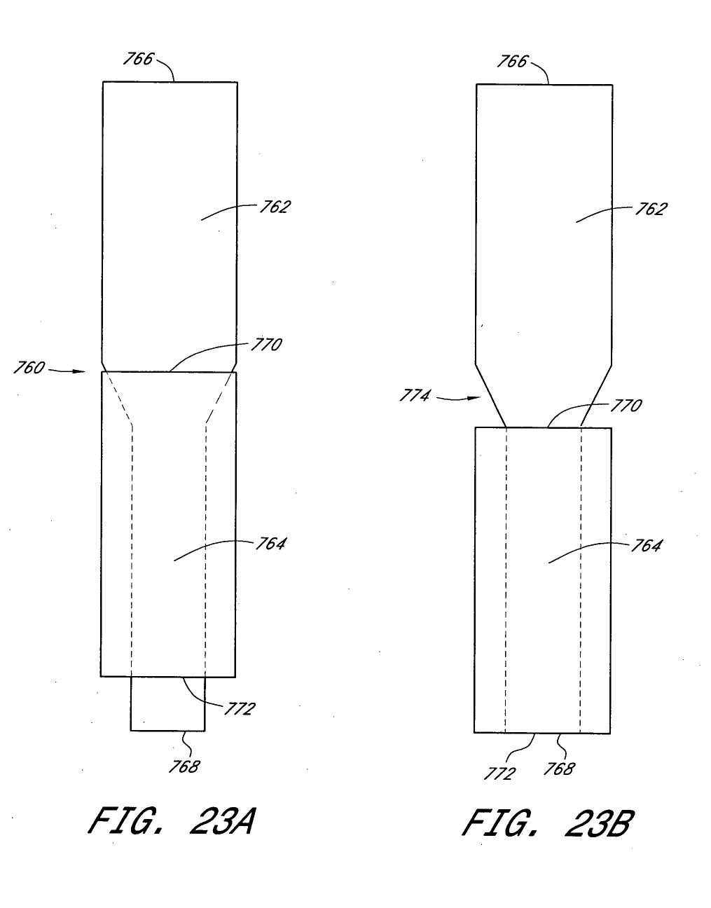

Referring to FIG. 21, another embodiment of a multilumen cannula 730 is similar to the cannula 700, except as set forth below. The cannula 730 includes a first elongate portion 732 defining a first lumen 734 and a second elongate portion 736 defining a second lumen 738, which lumens are shown in FIGS. 22A-22C.

The first elongate portion 732 extends between a first distal end 740 and a proximal end 744. The second elongate portion 736 extends between a second distal end 742 and the proximal end 744. The first distal end 740 of the first elongate portion 732 extends distally farther from the proximal end 744 of the multilumen cannula 730 than does the second distal end 742.

The multilumen cannula 730 includes a proximal portion 746, a transition portion 748, and a distal portion 756. In the proximal portion 746, the first and second elongate portions 732, 736 extend generally parallel to each other, and the first elongate portion 732 is coupled with the interior of the second elongate portion 736. In one embodiment, the first elongate portion 732 is attached to the second elongate portion 736 within the second lumen 738. In one embodiment, the first and second elongate portions 732, 736 form two non-concentric circles, one within another, as shown in FIG. 22C. The distal portion 756 of the cannula 730 has a cross-sectional size that is substantially the same as in the proximal portion 746. The interior cross-section shape of the first lumen 734 preferably is circular.

In the transition portion 748 of the cannula 730, the cross-sectional size of the first elongate portion 732 expands in a manner similar to the first elongate portion 702. Preferably the transition portion 748 provides an increase in size of the first elongate portion 732 such that at a location 750 distal the transition portion 748, the first elongate portion 732 has a outer size (e.g., an outer diameter) that is about the same as the outer size of the second elongate portion 736 at a location 752 proximal the second distal end 742. In one embodiment, the elongate portion 732 increases from about a seven French size in the proximal portion 746 to about a twelve French size in the distal portion 754. As shown in FIGS. 22A-22C, the cross-sectional shape of the lumen 734 preferably is circular at points within the proximal portion 746, the transition portion 748, and the distal portion 754. In one embodiment, the cross-sectional shape of the second lumen 734 is circular along the entire length of the first elongate portion 732.

With reference to FIGS. 22B and 22C, the inner cross-sectional size of the first lumen 734 expands compared to the inner-cross-sectional size of the first lumen 734 in the proximal portion 746 of the multilumen cannula 730 distal a location corresponding to the second distal end 742. In some embodiments, it is beneficial to provide at least about a one hundred percent increase in the size of the lumen 734 in the elongate portion 732 at the distal end 740 compared to the proximal end 744. The length of the transition portion 748 may be any suitable length, e.g., one that provides gradual increase distally to prevent abrupt changes in flow direction of the blood. In one embodiment, the length of the transition portion 748 is about one-half inch. In one embodiment, the length of the transition portion 748 is about one inch or less. In another embodiment, the length of the transition portion 748 is about one inch. The expanded size of the first lumen 734 through the transition portion 748 and in the distal portion 754 may make the inner cross-section of the first lumen 734 greater at the first distal end 740 than at the proximal end 744. As previously discussed, this configuration is advantageous in that the cannula 730 has lower flow resistance compared to a cannula of comparable length with a constant inner cross-sectional size equal to inner cross-sectional size of the proximal end of the cannula 730.

The multilumen cannula 730 is also configured in an advantageous manner for insertion into the vasculature of a patient. In the illustrated embodiment, both the proximal portion 746 and the distal portion 756 provide a substantially constant cross-sectional profile. As discussed above, the outer size of the multilumen cannula 730 is substantially the same at the location 752 and at the location 750. As discussed above in connection with FIG. 17, in some embodiments minimizing the length of the transition portion 748 is advantageous.

As discussed previously, it is desirable to design the cannula 730 so that the distal portion 754 comprises as large a fraction of the total length of the cannula as is possible, given other constraints on the cannula design.

Referring to FIGS. 23A-23B, another embodiment of a multilumen cannula 760 provides relative movement of two portions thereof. The cannula 760 is similar to the cannula 700 shown in FIGS. 19-20C, except as set forth below. The cannula 760 has a first elongate portion 762 and a second elongate portion 764. The first elongate portion 762 extends between a first distal end 766 and a first proximal end 768. The second elongate portion 764 extends between a second distal end 770 and a second proximal end 772. The first elongate portion 762 has a transition portion 774, wherein the first elongate portion 762 expands, as discussed above.

The first elongate portion 762 and the second elongate portion 764 of the cannula 760 are configured to translate relative to each other. In one embodiment, the first and second elongate portions 762, 764 are configured to couple in a manner that permits longitudinal translation. Longitudinal translation permits the first proximal end 768 and the second proximal end 772 to be positioned in a variety of positions such that the distances between the first and second proximal ends 768, 772 varies. As discussed more fully below, the relative motion advantageously permits the second distal end 770 to be positioned selectively at the same longitudinal position as the distal end of the transition portion (as shown in FIG. 23A) or at any suitable position proximally thereof. With reference to FIG. 23B, in one such position, the second distal end 770 is about at the same longitudinal location as the proximal end of the transition portion 774.

In some applications, the length of the cannulae hereinbefore described can be substantial. In such arrangements, flow resistance within the longer lumens can become significant. One detriment of increased flow resistance is a corresponding decreases in the flow (e.g., volumetric flow rate) at the distal end of the higher resistance lumen. One approach to maintain the flow at the distal end of the lumen is to increase the size of the lumen to overcome the flow reducing effect of flow resistance. However, the systems described herein often are deployed in relatively small vessels. For such applications, it is desirable to maintain the flow at the distal end of the lumen and to keep the cannulae relatively small. Reducing the resistance is one approach to maintain the flow at the distal end without greatly increasing the size of the cannulae. Another detriment of increased flow resistance is a corresponding increase in the power required to pump the blood through the cannulae. This increased power requirement may necessitate a larger pump, more frequent battery changes where the system is battery powered (e.g., for a portable system), or more frequent pump replacement. In many arrangements, e.g., where the pump is to be implanted into the patient, or the patient is desired to be ambulatory, it is desirable to minimize both the size and power consumption of the pump.

It is believed that power consumption can be reduced by reducing the flow resistance in these cannulae. The flow resistance of a cannula can be reduced by decreasing the overall length of the cannula, decreasing the viscosity of the fluid, or increasing the cross-sectional size of the cannula lumen or interior, as discussed above. The total cross-sectional size of the cannula is restricted by the size of the blood vessel into which the cannula is inserted. However, it is believed that an increase in the cross-sectional size of the lumens defined in the cannulae for at least a portion of the total length of the cannulae will result in a decrease in the overall flow resistance of the cannulae. Thus, the cannulae described herein are configured in this manner to reduce resistance to flow in relatively long lumens.

Reducing the resistance to the flow of blood in a lumen of a cannula can have additional benefits. For example, higher flow resistance in the lumen corresponds to a higher shear force being exerted on the blood flowing in the lumen. The exertion of higher shear force on the blood tends to increase the likelihood that the blood will be damaged, e.g., by hemolysis. Reducing the shear force being exerted on the blood tends to reduce the likelihood that the blood will be damaged, e.g., by hemolysis. The shear force being exerted on the blood advantageously may be reduced by reducing the resistance to blood flow in the lumen. As discussed herein, such flow resistance reduction may be accomplished by at least one of configuring the lumen to induce a rotation flow in the blood and increasing the size of at least a portion of the lumen.

Also, the longer the blood is subject to higher shear force, the greater the damage that may result to the blood. Accordingly, further benefit may be achieved by reducing the shear force being exerted on the blood for as much of the length of the lumen as possible. Accordingly, as discussed above, a greater benefit may be achieved by at least one of providing over as much of the lumen as possible a configuration that induces a rotational component in the flow of blood and by keeping the lumen as large as possible over most if not all of its length. Another benefit of keeping the lumen as large as possible and of reducing flow resistance is the resulting increase in the volume of flow in the lumen. Higher blood flow through the cannula(e) can increase the effectiveness thereof in a given treatment.

B. Cannula Assemblies for Insertion

The arrangement of the cannulae described above in connection with FIGS. 17-23B makes the cannulae difficult to apply to patients using traditional techniques. For example, these cannulae are generally incompatible with traditional dilators, because the longer lumen of each of the cannulae has a smaller cross-sectional area near the proximal end than near the distal end. A traditional dilator inserted through the proximal end of the longer lumen would have a cross-sectional area substantially smaller than that of the distal end of the longer lumen. The use of such a dilator will dilate tissue to provide for insertion of the cannulae and thus would not satisfactorily reduce trauma to the surrounding tissue.

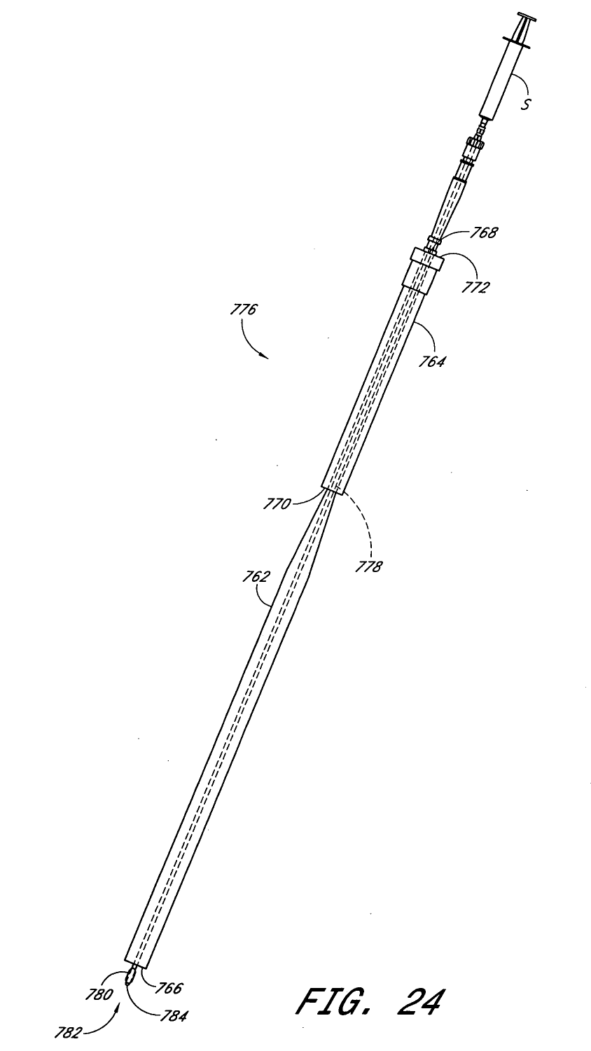

It is therefore desirable to provide cannula assemblies that include a cannula with a distally increasing lumen and a structure to ease insertion of the cannula. In one embodiment, a cannula assembly includes a dilator that is insertable through the distally increasing lumen and that can be configured after insertion into the lumen to reduce trauma to surrounding tissue during insertion of the cannula assembly. Thus, the cannulae described above in connection with FIGS. 17-23B may be applied percutaneously. A variety of embodiments of cannula assemblies including such dilators and methods of insertion of such assemblies, as well as operation of a heart assist systems incorporating such cannulae, are discussed below in connection with FIGS. 24-27.

With reference to FIG. 24, one embodiment of a percutaneous cannula assembly 776 includes the cannula 760, described above in connection with FIGS. 23A-23B, and a dilator 778. As discussed above, the cannula 760 includes a first elongate portion 762 and a second elongate portion 764. The first elongate portion 762 defines a first lumen (not shown) extending between a first distal end 766 and a first proximal end 768. The second elongate portion 764 defines a second lumen (not shown) extending between a second distal end 770 and a second proximal end 772. In one embodiment, a portion of the first elongate portion 762 extends through the second lumen. The dilator 778 of the percutaneous cannula assembly 776 extends through the first lumen, in one embodiment.

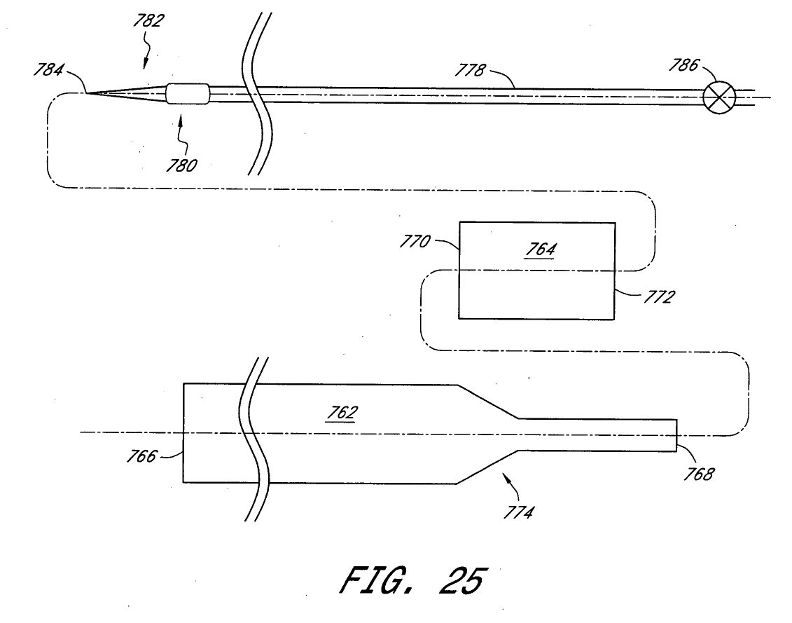

As shown in FIGS. 24-25, the dilator 778 includes a tip portion 782 located at a distal end 784 of the dilator 778. The tip portion 782 is configured to ease insertion of the cannula assembly 776 from outside the patient's body to a location within the vasculature. In one embodiment, the tip portion 782 comprises an expandable member 780 that extends proximally from the distal end 784 of the dilator 778. The expandable member 780, which may be a balloon, is shown in an un-expanded state in FIGS. 24-25. The dilator 778 is constructed such that it has sufficient rigidity to push tissue aside during insertion of the cannula assembly 776 into the vessel of a patient. In particular, the rigidity of the expandable member 780 is such that tissue will be compliantly moved aside as the dilator 778 is advanced therethrough.

In one embodiment, the dilator 778 includes an inflation lumen (not shown) in fluid communication with the expandable member 780. The inflation lumen may be further in fluid communication with a fluid pressure source, e.g., a syringe S as shown in FIG. 24 or other inflation means. The expandable member 780 may be inflated with saline solution or any other suitable liquid or gas. The dilator 778 may also include a valve 786 as shown in FIG. 25 in fluid communication with the inflation lumen. The valve 786 can take any suitable form. In one embodiment, the valve 786 can be actuated to a closed position after inflation fluid from the inflation means inflates the expandable member 780 and can be actuated to an open position to allow the inflation fluid in the expandable member 780 to escape from the expandable member 780. As discussed more fully below, removal of the inflation fluid causes the expandable member 780 to become un-expanded, e.g., deflated, to facilitate the removal of the dilator 780 from the portion of the lumen that extends through the proximal end 768 of the first elongate portion 762. Further details of features of cannula assemblies that may be combined with the assemblies discussed herein are set forth in U.S. Pat. No. 6,488,662, issued Dec. 3, 2002, which is hereby expressly incorporated by reference in its entirety and made a part of this specification.

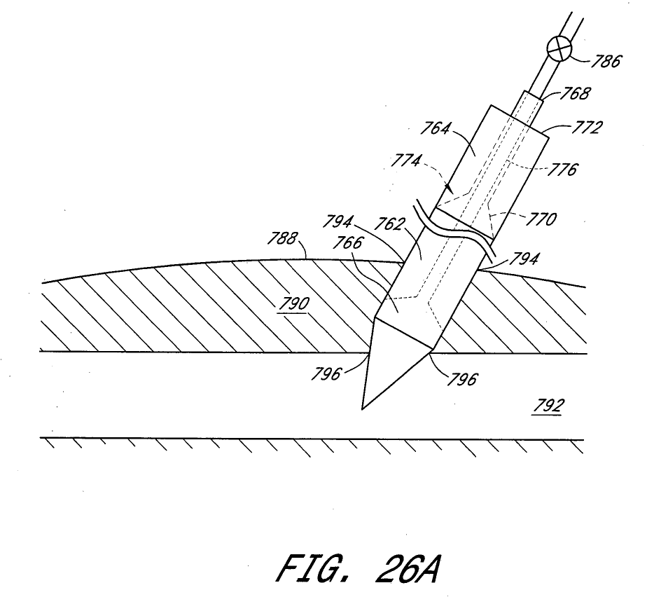

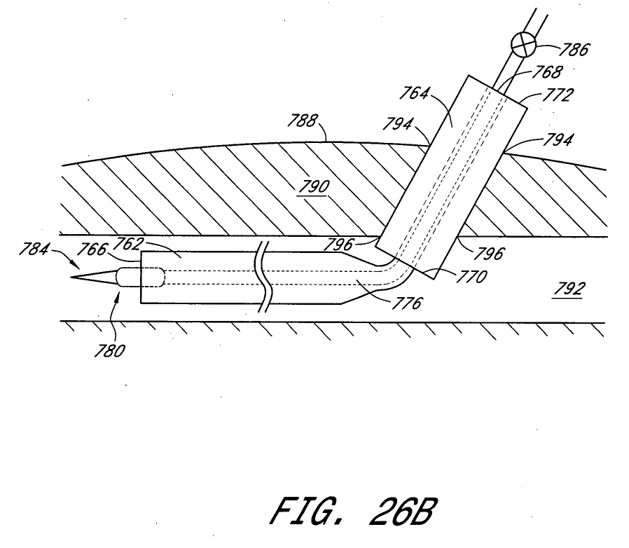

With reference to FIGS. 26A-26B, one method of inserting the cannula assembly 776 into a patient is provided. Prior to insertion of cannula assembly 776 into the vasculature of a patient, the second elongate portion 764 preferably is moved distally relative to the first elongate portion 762 until the distal end 770 is located distal at least a portion of the transition portion 774. The second distal end 770 is preferably located distal most if not all of the transition portion 774, as shown in FIG. 26A. This corresponds to a first position of the cannula 760.

Prior to insertion of the percutaneous cannula assembly 776 into the vasculature of a patient, the expandable member 780 is actuated to an expanded state. In one embodiment, saline solution is forced into the inflation lumen of the dilator 778. Once the expandable member 780 has been sufficiently expanded, the valve 786, where provided, may be closed. In another method, a gas or other liquid is used to expand the expandable member 780. The dilator 778 may then be disconnected from the syringe or other inflation means so that the operator can more easily manipulate the cannula assembly 776.

When the dilator 778 is in the expanded state, the cannula 760 is advantageously configured to be advanced through the patient's skin, through tissue beneath the skin, and into the vasculature. For example, the dilator 778 is configured so as to minimize any exposed transition between the expandable member 780 and the distal end 766 of the first elongate portion 762. Such an exposed transition could become hung up on tissue 790 between the skin and a vessel 792 or on the vessel itself during insertion, which could result in trauma to the tissue 790 or vessel 792. Furthermore, providing minimal transition between the dilator 778 and the first elongate portion 762 in the cannula assembly 776 can reduce the risk of damage to the interior of the vessel 792 or of dislodging of material attached to the vessel wall.

As discussed above, the outer cross-sectional size of the first elongate portion 762 and the outer cross-sectional size of the second elongate portion 764 are substantially equal in one embodiment. By configuring the cannula 760 for movement to the first position (shown in FIG. 26A), the cannula 760 is further advantageously configured for insertion into the vasculature of a patient. In particular, the exposed portion of the transition portion 774, which might provide a discontinuity in the outer cross-sectional size of the cannula 760 along its length, is reduced or eliminated. The second distal end 770 of the second elongate portion 764 is beveled in one embodiment, e.g., at an angle equal to the taper of the transition section 774, so that when the cannula 760 is in the first position, a flush contact between the first and second elongate portions 762, 764 is obtained. One skilled in the art would understand that there are other ways to minimize of eliminate a discontinuity between the first and second elongate portions 762, 764. Elimination of the discontinuity can provide advantages during insertion or removal of the cannula assembly 776, as discussed above.

While placement of the cannula 760 in the first position prior to inflation or expansion of the expandable member 780 is preferred, it is to be understood by one skilled in the art that inflation of the expandable member 780 can alternately take place before the cannula 760 is placed in the first position. In another application, the cannula assembly 776 can be partially inserted, e.g., up to the location of the transition section 774 before the cannula 760 is translated to the first position, which is shown in FIG. 26A.

Further advantages of inserting the cannula assembly 776 in the first position is that contact between the first elongate portion 762 and the second distal end 770 of the second elongate portion 764 may advantageously minimize or eliminate unwanted blood-flow into the second elongate portion 764 at the second distal end 770 during insertion. Also, contact between the first elongate portion 762 and the second distal end 770 of the second elongate portion 764 may prevent or minimize the likelihood of introduction of emboli (e.g., gaseous or particulate) into the cannula 760 and possibly into the patient.

With reference to FIG. 26B, a further step of one method of applying the cannula assembly 776 involves inserting the cannula assembly 776 through the skin 788 of a patient at a percutaneous insertion site 794, and partially into a vessel 792 through a vascular insertion site 796. The second elongate body 764 defines a wall engagement portion, e.g., a portion of the outer surface of the wall of the second elongate body 764 that engages the wall of the blood vessel 792 where the cannula 760 passes from the tissue 790 into the vessel 792. In one embodiment, the inner cross-section of the first lumen is greater at the first distal end 766 than at a location corresponding to the vascular wall engagement portion, as shown in FIG. 26A.

In the illustrated embodiment, the expandable member 780 extends partially into the first lumen at the distal end 766. In addition to easing insertion into the blood vessel 792 at the vascular insertion site 796, blood-flow into the first elongate portion 762 through the first distal end 766 is minimized or eliminated when the expandable member 780 is inflated. Other embodiments of the cannula assembly 776 may make use of an expandable member 780 fully distal the distal end 766 of the first elongate portion 762, or a dilator 778 without an inflatable expandable member 780 (e.g., a dilator where the tip comprises an umbrella-like structure, capable of opening without inflation).

Further steps of various applications of the cannula assembly 776 to the blood vessel 792 may be performed after the percutaneous cannula assembly 776 has been inserted into the vessel 792, as shown in FIG. 26B. In particular, after the second distal end 770 is located fully within the vessel 792, the first elongate portion 762 can be translated relative the second elongate portion 764 so that the cannula 760 is in a second position, as shown in FIG. 26B. In the second position, at least a portion of the transition portion 774 is located distal the second distal end 770 so that the transition portion 774 is exposed and blood-flow through the second distal end 770 is permitted. The length of the second elongate portion 764 is selected such that when the cannula 760 is in the second distal position, the second distal end 770 is in a desired location for inflow of blood to a pump and the first distal end 766 is in a desired location for outflow of blood from the pump. In one method, when the cannula 760 is in the second position, the second proximal ends 768, 772 are approximately flush with one another. This arrangement is advantageous, in that alignment of the first and second proximal ends 768, 772 will provide an indication of the location of the first and/or the second distal ends 766,770. This arrangement will facilitate moving the first elongate portion 762 to the proper position while the cannula 760 is inserted into a vessel 792 of the patient and therefore not visible.

It is also contemplated that the second elongate portion 764 may be translated, rather than the first elongate portion 762. In such a case, the cannula 760 must be inserted into the vessel 792 so that the entire transition portion 774 is located within the vessel 778. During translation of the second elongate portion 764, care should be taken that the second distal end 770 is not moved proximal the cannulation site 794. Selection of the length of the second elongate portion 764 so that the proper second position is apparent from the relative locations of the proximal ends 768, 772 is therefore advantageous. Translation of the first elongate portion 762 may be advantageous in that it may reduce the likelihood of damage to the walls of the vessel 792 because the first elongate portion 762 is not in contact with the wall of the vessel 792 at the vascular insertion point 796.

After insertion of the distal end 766 of the first elongate portion 762 of the percutaneous cannula assembly 776 into the vessel 792, the expandable member 780 may be un-expanded, e.g., deflated. In one embodiment, deflation may occur upon the opening of the valve 796. In another embodiment, un-expansion of the expandable member 780 may require use of a syringe, pump, or other source of negative pressure to aspirate the inflation medium. Once the expandable member 780 is in the un-expanded state, a transverse annulus is provided between the outer surface of the expandable member 780 and the first elongate portion 762 through which blood-flow through the first distal end 766 into the lumen is permitted (see FIG. 26B).

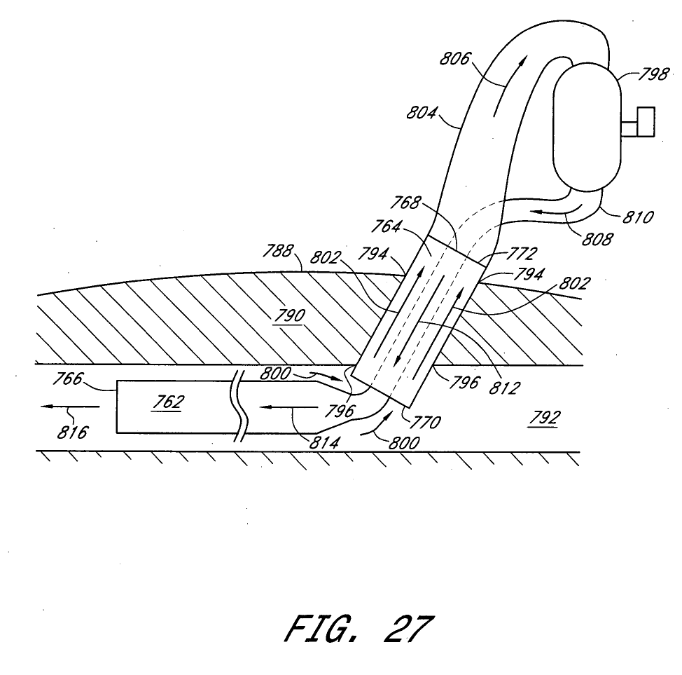

At some point after the un-expansion of the expanding member 780, the dilator 778 can be withdrawn proximally from the cannula 760 through the first lumen. In one method, once the cannula 760 is actuated to the second position, and the dilator 778 is removed, the proximal ends 768, 772 are placed in fluid communication with a pump 798, as shown in FIG. 27.

With continued reference to FIG. 27, operation of an extracardiac system is illustrated by a series of arrows. In particular, arrows 800 indicate that blood in the vessel 792 may be drawn through the second distal end 770 of the cannula 760. In one embodiment, the second lumen defined between the second distal end 770 and the second proximal end 772 is generally annular in cross-section, e.g., defined by the inner surface of the second elongate portion 764 and the outer surface of the first elongate body 762. Other arrangements of the second lumen are discussed above. Arrows 802 illustrate blood flow in the second lumen. In this application, the blood flow indicated by the arrows 802 is proximally, toward the pump 798. The blood flow is conveyed thereafter from the second proximal end 772 of the cannula 760 by way of a conduit 804, though any other suitable conveyance may be provided. Arrow 806 illustrates blood flow in the conduit 804.

The pump 798 may be any suitable pump, e.g., any of those discussed above. In particular, the pump 798 may be one that is configured to pump blood at subcardiac rates. The pump 798 may also be implanted subcutaneously, e.g., just beneath the skin or within the vasculature, as discussed above. Arrow 808 indicates blood flow that is conveyed from the pump 798 to the first proximal end 768 of the first elongate body 762 through a conduit 810 or other suitable conveyance. Blood flow enters the first lumen at the first proximal end 768 and flows distally therein, as indicated by arrow 812 to the transition portion 774. The transition portion 774 corresponds to an area of the first lumen wherein the effect of resistance to blood-flow is lessened, as discussed more fully below. The cannula 760 lessens the effect of resistance by providing an increase in the cross-sectional area of the first lumen through the transition portion 774, which may extend partially or all the way to the first distal end 766. The blood flow distal the distal end of the transition portion 774 to the first distal end 766 is illustrated by arrow 814. In the illustrated embodiment, the first distal end 766 is configured to expel blood from the first lumen distally into the blood vessel 792 (or into another blood vessel) generally in the same direction as that of the blood flow in the first lumen, as indicated by arrow 816. A variety of arrangements may be provided on cannula 760, or on the distal end of any of the cannulae described herein, to provide beneficial flow characteristic at the distal end. Some such arrangements are set forth in U.S. patent application Ser. No. 10/706,346, filed Nov. 12, 2003, entitled CANNULAE HAVING REDIRECTING TIP, incorporated by reference hereinabove.

While the translatable aspect of the elongated portions was described in connection with the cannula 760, the cannulae 666 and 730 can be arranged such that the first and second elongate portions are translatable relative to one another, as well. Any suitable translatable structure may be provided. For example, with reference to FIG. 17, the cannula 666 may comprise a D-shaped structure comprising the first elongate portion 668 and the wall 684 and a C-shaped second elongate portion 672. The C-shaped structure may be attached to the D-shaped structure by means of two tongue-and-groove structures, running from the proximal end 680 of the D-shaped structure to a location within or distal the transition portion 690, permitting the C-shaped second elongate portion to slide relative to the D-shaped structure. The C-shaped structure may thus be moved to a first position, partially or completely inhibiting the flow of blood through the second distal end 678. After insertion, the C-shaped elongate portion 672 may be moved to a second position, permitting the flow of blood through the second distal end 678. Other arrangements are also possible.

Similarly, with reference to FIG. 21, the multilumen cannula 730 may be arranged for translatable movement in any suitable manner. In one embodiment, the first elongate portion 732 of the cannula 730 is connectable to the second elongate portion 736 by means of a tongue-and-groove structure running from the second proximal end 744 to the second distal end 742 of the second elongate portion 736. The second elongate portion 736 may thus be moved to a first position configured for insertion to a second position after insertion configured for operation of the cannula 730 within a system similar to those hereinbefore described.

One skilled in the art will recognize that the cross-sectional size of the proximal portion of the cannula outside of the vessel need not be limited by the cross-sectional size of the blood vessel. Thus, any of the multilumen cannulae 666, 700, 730, 760, or any other embodiments can have inner cross-sectional sizes at their proximal end that are greater than their cross-sectional sizes at their distal ends. Nevertheless, the cross-sectional size of the first lumen preferably will be greater at the distal end than at the vascular insertion site, as the vascular insertion site is the most proximal point at which the cross-sectional size of the cannula is limited by the size of the blood vessel.

As discussed above, the cannula 760 may be applied in the vasculature in the vessel 792 (see FIGS. 26B and 27), which may correspond to any of a wide variety of vascular locations. In various applications, the cannula 760, as well as the other cannulae described herein, may be inserted into the vasculature through a vascular insertion site located in a peripheral vessel or in a non-primary vessel, e.g., a peripheral or non-primary artery or vein. Cut lines extend through the first elongate portion 762 to indicate that the length of the first elongate portion 762 varies as required. For example, it may be desirable to apply the cannula 760 such that the first distal end 766 resides in any of a wide range of vessels, including the aorta, the vena cava, and any peripheral or non-primary artery or vein, including the renal or any other branch artery, as discussed above.

Although the foregoing invention has been described in terms of certain preferred embodiments, other embodiments will be apparent to those of ordinary skill in the art. Additionally, other combinations, omissions, substitutions and modification will be apparent to the skilled artisan, in view of the disclosure herein. Accordingly, the present invention is not intended to be limited by the recitation of the preferred embodiments, but is instead to be defined by reference to the appended claims.

1. A cannula system comprising:
a multilumen cannula comprising:
a first elongate body comprising a proximal portion, a transition portion distal of the proximal portion, the transition portion having a diameter at a distal end greater than a diameter at a proximal end, a distal portion distal of the transition portion, and a first lumen that extends through the first elongate body, the first lumen having a first cross-sectional area within the proximal portion and a second cross-sectional area within the distal portion, the second cross-sectional area being greater than the first cross-sectional area; and
a second elongate body defining a second lumen, the second elongate body extending along the proximal portion of the first elongate body; and
a dilator configured to extend through the first lumen.

2. The cannula system of claim 1, wherein the distal portion of the dilator comprises an expandable member for dilating tissue to facilitate insertion of the multilumen cannula and a low-profile configuration for withdrawing the dilator from the first lumen.

3. The cannula system of claim 2, wherein the expandable member is a balloon.

4. The cannula system of claim 2, wherein the dilator further comprises a valve configured to maintain the expandable member in an expanded state.

5. The cannula system of claim 2, wherein the dilator further comprises a lumen in fluid communication with the expandable member.

6. The cannula system of claim 1, wherein the second elongate body is translatable relative to the first elongate body.

7. The cannula system of claim 1, wherein the first elongate body further comprises means for creating substantially vortex flow in the first lumen.

8. The cannula of claim 7, wherein the vortex flow creating means comprises a ridge configured to protrude inwardly into the first lumen, the ridge having a helical arrangement.

9. The multilumen cannula of claim 1, wherein the first elongate body further comprises a ridge configured to protrude inwardly into the first lumen, the ridge having a helical arrangement.

10. A cannula system comprising:
a multilumen cannula comprising:
a first elongate body comprising a proximal portion, a distal portion, and a first lumen that extends through the first elongate body, the first lumen being configured to reduce resistance to flow therethrough; and
a second elongate body defining a second lumen, the second elongate body extending along the proximal portion of the first elongate body; and
a dilator configured to extend through the first lumen.

11. The cannula system of claim 10, wherein the first lumen has a first cross-sectional area within the proximal portion and a second cross-sectional area within the distal portion, the second cross-sectional area being greater than the first cross-sectional area

12. The cannula system of claim 10, wherein the first elongate body further comprises means for creating substantially vortex flow in the first lumen.

13. The cannula system of claim 12, wherein the vortex flow creating means comprises a ridge configured to protrude inwardly into the first lumen, the ridge having a helical arrangement.

14. The cannula system of claim 10, wherein the first elongate body further comprises a ridge configured to protrude inwardly into the first lumen, the ridge having a helical arrangement.

15. A method for inserting a cannula into a blood vessel, comprising:
providing a multilumen cannula having a first elongate body and a second elongate body, the first elongate body comprising a proximal portion and a distal portion and defining a first lumen extending therethrough, the first lumen having a larger cross-sectional area in the distal portion than in the proximal portion, the second elongate body defining a second lumen extending therethrough;
advancing a dilator into the first lumen in the proximal portion of the first elongate body until a distal portion of the dilator extends distal of the distal portion of the first elongate body;
actuating the distal portion of the dilator until the cross-sectional area of the distal portion of the dilator is about equal to the cross-sectional area of the first elongate body at a distal end of the distal portion; and
advancing the dilator and the cannula through a percutaneous insertion site and through a vascular insertion site into a blood vessel.

16. The method of claim 15, further comprising withdrawing the dilator from the first lumen of the cannula.

17. The method of claim 15, further comprising deactuating the distal portion of the dilator.

18. The method of claim 17, further comprising withdrawing the dilator from the first lumen of the cannula.

19. The method of claim 15, wherein step of actuating the distal portion comprises inflating a balloon located at the distal end of the dilator.

20. The method of claim 19, wherein the first elongate body further comprises means for creating substantially vortex flow in the first lumen.

21. The method of claim 20, wherein the vortex flow creating means comprises a ridge configured to protrude inwardly into the first lumen, the ridge having a helical arrangement.

22. The method of claim 15, wherein the first elongate body further comprises a ridge configured to protrude inwardly into the first lumen, the ridge having a helical arrangement.

23. A method for treating a patient, comprising:
providing a cannula system comprising:
a multilumen cannula having a first elongate body comprising a proximal portion and a distal portion and defining a first lumen extending therethrough, the first lumen being configured to reduce resistance to flow therethrough, and a second elongate body defining a second lumen; and
a dilator extending through the first lumen;
advancing the cannula system into a blood vessel through a vascular insertion site;
providing fluid communication between the second lumen and an inflow port of a pump and between the first lumen and an outflow port of the pump; and
operating said pump to perfuse blood through the patient's circulatory system.

24. The method of claim 23, further comprising configuring a distal portion of the dilator such that a cross-sectional area thereof is about equal to the cross-sectional area of the first elongate body in the distal portion.

25. The method of claim 23, further comprising translating the second elongate body distally relative to the first elongate body prior to advancing the cannula system into the blood vessel.

26. The method of claim 25, further comprising translating the second elongate body proximally relative to the first elongate body after advancing the cannula system into the blood vessel.

27. The method of claim 23, further comprising translating the first elongate body proximally relative to the second elongate body prior to advancing the cannula system into the blood vessel.

28. The method of claim 27, further comprising translating the first elongate body distally relative to the second elongate body after advancing the cannula system into the blood vessel.

29. The method of claim 23, wherein the pump is operated to perfuse blood through the patient's circulatory system at volumetric rates that are on average subcardiac.

30. The method of claim 23, wherein advancing the cannula system further comprises advancing the cannula system through a vascular insertion site of a non-primary blood vessel.

31. The method of claim 30, wherein advancing the cannula system into a blood vessel further comprises advancing the distal end of the first elongate body into a branch artery.

32. The method of claim 31, wherein advancing the cannula system further comprises advancing the distal end of the first elongate body into a renal artery.

33. The method of claim 23, wherein advancing the cannula system further comprises advancing the distal end of the first elongate body into the aorta.

34. The method of claim 33, wherein advancing the cannula system further comprises advancing the distal end of the first elongate body into the thoracic aorta.

35. The method of claim 23, wherein the first lumen has a larger cross-sectional area in the distal portion than in the proximal portion.

36. The method of claim 23, wherein the first elongate body further comprises means for creating substantially vortex flow in the first lumen.

37. The method of claim 36, wherein the vortex flow creating means comprises a ridge configured to protrude inwardly into the first lumen, the ridge having a helical arrangement.

38. The method of claim 23, wherein the first elongate body further comprises a ridge configured to protrude inwardly into the first lumen, the ridge having a helical arrangement.
